(19) United States
(12) Patent Application Publication
(10) Pub. No.: US 2024/0033477 A1
(43) Pub. Date: Feb. 1, 2024

(54) LONG-LIVED INSULIN DELIVERY CANNULA

(71) Applicant: Pacific Diabetes Technologies Inc, Portland, OR (US)

(72) Inventors: William Kenneth Ward, Portland, OR (US); Robert S. Cargill, Portland, OR (US); Thomas L. Seidl, Wilsonville, OR (US); Solomon REID, Portland, OR (US)

(21) Appl. No.: 18/212,477

(22) Filed: Jun. 21, 2023

Related U.S. Application Data
(63) Continuation of application No. PCT/US2021/064448, filed on Dec. 20, 2021.
(60) Provisional application No. 63/128,836, filed on Dec. 21, 2020.

(51) Int. Cl. A61M 25/00; A61K 38/28; A61K 47/10

(52) U.S. Cl. CPC A61M 25/007; A61K 38/28; A61K 47/10; A61M 2202/0445

(57) ABSTRACT

Described herein are systems and methods for creating a transport tube comprising a plurality of passages configured to deliver an composition from a source into the human subcutaneous space.

### CROSS-REFERENCE

This application is a continuation of International Application No. PCT/US2021/064448, filed Dec. 20, 2021, which claims the benefit of U.S. Provisional Application No. 63/128,836, filed Dec. 21, 2020, each of which is incorporated by reference herein in its entirety.

### BACKGROUND

Insulin-using patients often use insulin pumps to deliver insulin on a continuous basis, a process known as continuous subcutaneous insulin infusion (CSII). Traditional pumps have a length of plastic tubing that connect the pump reservoir to the subcutaneous cannula. On-the-body pumps, also known as patch pumps, are secured to the skin with the aid of an adhesive and attach directly to the subcutaneous cannula without the need for a connecting tube. The element of the pumping system that limits the use time to 72 hours is the subcutaneous cannula. In fact, Heinemann has described the insulin cannula as the Achilles Heel of CSII (Heinemann, L. and Krinelke, L., Insulin Infusion Set: The Achilles Heel of Continuous Subcutaneous Insulin Infusion, J Diabetes Sci Technol. 2012 July; 6(4):954-964).

Though some users keep a cannula in place for longer periods of time, they are approved by the FDA for no more than 72 hours. The most common reason that limits the use life of a cannula use is occlusion. As noted by Joseph et al., there are several causes of occlusion, including blood clot (a clot includes fibrin and platelets), white blood cell clumping, deposition of collagen, the forcing of subcutaneous tissue into the distal lumen tip during insertion, and kinking of the cannula itself. (Eisler G, Kastner J R, Torjman M C, et al., In vivo investigation of the tissue response to commercial Teflon insulin infusion sets in large swine for 14 days: the effect of angle of insertion on tissue histology and insulin spread within the subcutaneous tissue, BMJ Open Diabetes Res Care, 2019 December; 7(1):e000881, and Jasmin R. Hauzenberger, Brian R. Hipszer, Channy Loeum, et al., Detailed Analysis of Insulin Absorption Variability and the Tissue Response to Continuous Subcutaneous Insulin Infusion Catheter Implantation in Swine, Diabetes Technology & Therapeutics, November 2017; 641-650.)

One method of mitigating the distal cannula occlusion is to place at least one hole in the side wall of the cannula. The concept here is that if occlusion occurs at the distal site, the insulin composition can nonetheless reach the subcutaneous space via the side hole, as described in a human study by Gibney et al. (Gibney M, Xue Z, Swinney M, Bialonczyk D, Hirsch L., Reduced Silent Occlusions with a Novel Catheter Infusion Set (BD FlowSmart): Results from Two Open-Label Comparative Studies, Diabetes Technol Ther., 2016 March; 18(3):136-43). Such a device, known as the FlowSmart catheter, was designed, manufactured, and sold. However, it was removed from the market soon after its product launch due to kinking. One problem with this device was a weakness in the wall at the site of the hole that predisposed to formation of a kink.

There is an unmet need for continued delivery of an insulin composition when the distal port is occluded. To avoid kinking, the solution(s) must avoid mechanical weakening of the cannula shaft.

### SUMMARY

In one aspect, disclosed herein are systems comprising an transport tube configured to deliver a composition from a source into a subcutaneous space, the transport tube comprising: a distal passageway configured to reside in the subcutaneous space comprising: a distal tip, wherein the distal tip is open when it is first placed into the subcutaneous space; and a plurality of passages connecting a lumen of the transport tube with an outside surface of the transport tube, wherein the plurality of passages are configured to deliver the composition into the subcutaneous space when the distal tip is occluded, thereby allowing the transport tube to reside in the subcutaneous space for at least four days; and a proximal solid segment configured to reside on or above a skin surface, wherein the distal passageway and the proximal solid segment are in fluid communication. In some embodiments, the transport tube is configured to reside in the subcutaneous space for at least seven days. In some embodiments, transport tube is configured to reside in the subcutaneous space for at least ten days. In some embodiments, plurality of passages are a plurality of through-passages. In some embodiments, a through-passage of the plurality of through-passages is not in fluid communication with one another. In some embodiments, the plurality of passages are a plurality of interconnected passages. In some embodiments, an interconnected passage of the plurality of interconnected passages is in fluid communication with one another. In some embodiments, the composition comprises insulin. In some embodiments, the composition comprises fast-acting insulin, intermediate-acting insulin, or long-acting insulin. In some embodiments, the composition comprises fast-acting insulin. In some embodiments, when the distal tip of the transport tube is occluded in the subcutaneous space, a positive pressure builds up in the lumen of the transport tube. In some embodiments, the positive pressure in the lumen of the transport tube is at least 1 PSI. In some embodiments, the composition comprises at least one pharmaceutical acceptable excipient. In some embodiments, the at least one pharmaceutical acceptable excipient comprises phenol, cresol, a salt, a stabilizing agent, or any combination thereof.

In another aspect, disclosed herein are system comprising an transport tube configured to deliver a composition from a source into a subcutaneous space, the transport tube comprising: a distal passageway configured to reside in the subcutaneous space comprising: a distal tip, wherein the distal tip is open when it is first placed into the subcutaneous space; and a plurality of passages connecting a lumen of the transport tube with an outside surface of the transport tube, wherein a passage of the plurality of passages comprises a diameter, wherein the diameter is sufficiently small to prevent a flow of the composition through the passage into the subcutaneous space when the distal tip is open and is sufficiently large to deliver the composition through the passage into the subcutaneous space during an occlusion of the distal tip; and a proximal solid segment configured to reside on or above a skin surface, wherein the distal passageway and the proximal solid segment are in fluid communication. In some embodiments, the diameter is about 40 to 100 micrometers. In some embodiments, the diameter is about 40 to 80 micrometers. In some embodiments, the diameter is about 40 to 60 micrometers. In some embodiments, plurality of passages is a plurality of through-passages. In some embodiments, a through-passage of the plurality of through-passages is not in fluid communication with one another. In some embodiments, the plurality of passages is a plurality of interconnected passages. In some embodiments, an interconnected passage of the plurality of interconnected passages is in fluid communication with one another. In some embodiments, the composition comprises insulin. In some embodiments, the composition comprises fast-acting insulin, intermediate-acting insulin, or long-acting insulin. In some embodiments, the composition comprises fast-acting insulin. In some embodiments, when the distal tip of the transport tube is occluded in the subcutaneous space, a positive pressure builds up in the lumen of the transport tube. In some embodiments, the positive pressure in the lumen of the transport tube is at least 1 PSI. In some embodiments, the composition comprises at least one pharmaceutical acceptable excipient. In some embodiments, the at least one pharmaceutical acceptable excipient comprises phenol, cresol, a salts, a stabilizing agent, or any combination thereof.

In another aspect, disclosed herein are system comprising a transport tube configured to deliver a composition from a source into a subcutaneous space, the transport tube comprising: a distal passageway configured to reside in the subcutaneous space comprising: a distal tip, wherein the distal tip is open when it is first placed into the subcutaneous space; and a plurality of passages connecting a lumen of the transport tube with an outside surface of the transport tube, wherein a wall of the distal passageway comprises an empty fraction sufficiently low to prevent a kink in the transport tube; and a proximal solid segment configured to reside on or above a skin surface wherein the distal passageway and the proximal solid segment are in fluid communication. In some embodiments, the empty fraction is a percentage of area taken up by the plurality of passages in the wall of the distal passageway over total area of the wall of the distal passageway. In some embodiments, the empty fraction is about 5% to 75%. In some embodiments, the empty fraction is about 5% to 55%. In some embodiments, the empty fraction is about 5% to 25%. In some embodiments, plurality of passages are a plurality of through-passages. In some embodiments, a through-passage of the plurality of through-passages is not in fluid communication with one another. In some embodiments, the plurality of passages are a plurality of interconnected passages. In some embodiments, an interconnected passage of the plurality of interconnected passages is in fluid communication with one another. In some embodiments, the composition comprises insulin. In some embodiments, the composition comprises fast-acting insulin, intermediate-acting insulin, or long-acting insulin. In some embodiments, the composition comprises fast-acting insulin. In some embodiments, when the distal tip of the transport tube is occluded in the subcutaneous space, a positive pressure builds up in the lumen of the transport tube. In some embodiments, the positive pressure in the lumen of the transport tube is at least 1 PSI. In some embodiments, the composition comprises at least one pharmaceutical acceptable excipient. In some embodiments, the at least one pharmaceutical acceptable excipient comprises phenol, cresol, a salts, a stabilizing agent, or any combination thereof.

In a further aspect, disclosed herein are methods of creating a through-passage in a transport tube, the method comprising: (a) solvating a polymer in a solvent; (b) forming the polymer into a sheet, thereby creating a polymer sheet; (c) evaporating the solvent by raising the temperature of the polymer sheet; (d) making a longitudinal seam in a portion of the polymer sheet in order to create a tubular shape; and (e) creating a through-passage by application of a laser either after (c) or (d). In some embodiments, the polymer is a polyurethane. In some embodiments, the solvent has a boiling point is below 110 degrees C. In some embodiments, the solvent is tetrahydrofuran. In some embodiments, (c) comprises raising the temperature to at least 50 degrees C. In some embodiments, (d) comprises welding a portion of the polymer sheet longitudinally to create the longitudinal seam. In some embodiments, the laser comprises a yttrium garnet aluminum garnet (YAG) laser, carbon dioxide laser, excimer lasers, nonlinear mixing lasers, ion lasers, neutral atom lasers, or a combination thereof. In some embodiments, the through-passage connects a lumen of the transport tube with an outside surface of the transport tube. In some embodiments, the through-passage a diameter of about 40 micrometers to about 100 micrometers. In some embodiments, the through-passage does not connect with another through-passage. In some embodiments, the through-passage is created in a distal passageway of the transport tube. In some embodiments, the distal passageway segment has an empty fraction of about 5% to about 75%. In some embodiments, the distal passageway is configured to reside in the subcutaneous space. In some embodiments, the distal passageway is coupled to a proximal solid segment. In some embodiments, the proximal solid segment does not comprise a through-passage. In some embodiments, the solid tubular segment is configured to reside on or above the skin surface.

In a further aspect, disclosed herein are methods of creating interconnected passages in a transport tube, the method comprising: (a) adding a porogen compound to a polymer resin to create a mixture; (b) heating said mixture to above a melting point or glass transition point of the polymer resin; and (c) forcing molten said mixture though a die to create a tubular shape. In some embodiments, the porogen is a gas-forming compound of the nature X(HCO₃)_{Y} wherein X is ammonium or an element found in Column I or II of the periodic table of elements, wherein and Y is 1 if X is ammonium or the element found in Column I, wherein Y is 2 if X is the element found in Column II. In some embodiments, X is Na and Y is 1. In some embodiments, the porogen is a non-gas-forming compound or a collection of polymer microspheres. In some embodiments, the interconnected passages connect a lumen of the transport tube with an outside surface of the transport tube. In some embodiments, the interconnected passage has a diameter of about 40 micrometers to about 100 micrometers. In some embodiments, the interconnected passage is created in a distal passageway of the transport tube. In some embodiments, the distal passageway segment has an empty fraction of about 5% to about 75%. In some embodiments, the distal passageway is configured to reside in the subcutaneous space. In some embodiments, the distal passageway is coupled to a proximal solid segment. In some embodiments, the proximal solid segment does not comprise interconnected passages. In some embodiments, the proximal solid segment is configured to reside on or above the skin surface.

In another further aspect, disclosed herein are methods for delivering a composition to a subcutaneous space in a subject to treat a disease or disorder, comprising: providing a transport tube, wherein the transport tube comprises a distal passageway configured to reside in the subcutaneous space and a proximal solid segment configured to reside on or above a skin surface, wherein the distal passageway and the proximal solid segment are in fluid communication; inserting the transport tube into the subcutaneous space; and delivering a sufficient amount of the composition through the transport tube into the subcutaneous space, wherein when a distal tip of the distal passageway becomes occluded in the subcutaneous space; or when the distal tip is not occluded in the subcutaneous space. In some embodiments, the disease or disorder comprises insulin resistance. In some embodiments, the disease or disorder comprises a Type 1 diabetes mellitus. In some embodiments, the disease or disorder comprises a Type 2 diabetes mellitus. In some embodiments, the method further comprises replacing the transport tube at least 4 days after an insertion of the transport tube into the subcutaneous space. In some embodiments, the method further comprises replacing the transport tube at least 7 days after an insertion of the transport tube into the subcutaneous space. In some embodiments, the method further comprises replacing the transport tube at least 10 days after an insertion of the transport tube into the subcutaneous space. In some embodiments, the composition comprises insulin. In some embodiments, the composition comprises fast-acting insulin, intermediate-acting insulin, or long-acting insulin. In some embodiments, the composition comprises fast-acting insulin. In some embodiments, the composition further comprises at least one pharmaceutical acceptable excipient. In some embodiments, the at least one pharmaceutical acceptable excipient comprises phenol, cresol, a salts, a stabilizing agent, or any combination thereof.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a method of extruding resin into tubes (creating a cannula) and controlling tube size (diameter and wall thickness) in a vacuum tank, in accordance with some embodiments;

FIG. 2 shows a non-limiting example of a polymer sheet with laser-ablated through-passages, in accordance with some embodiments;

FIG. 3A shows a non-limiting example of through-passages in a cannula wall immediately after insertion into subcutaneous tissue, in accordance with some embodiments;

FIG. 3B shows a non-limiting example of impeded flow via through-passages in a cannula wall after at least several days in subcutaneous tissue, in accordance with some embodiments;

FIG. 4A shows a non-limiting example of interconnected passages in a cannula wall immediately after insertion, in accordance with some embodiments;

FIG. 4B shows a non-limiting example of a substantial maintenance of flow in interconnected passages in a cannula wall after several days in subcutaneous tissue, in accordance with some embodiments;

FIG. 5 shows a non-limiting example of a hole laser-ablated through the wall of a polyimide tube, in accordance with some embodiments;

FIG. 6 shows a photograph of an experiment in which dyed water can be seen escaping into air from a polyimide tube, in accordance with some embodiments; and

FIG. 7 shows a photograph of an experiment in which dyed water can be seen escaping into water from a polyimide tube immersed in the water, in accordance with some embodiments.

### DETAILED DESCRIPTION

The present disclosure provides solutions to create a stable and long-lasting cannula for delivering suitable pharmaceutical compositions, such as insulin. A plurality of passages in the cannular wall is designed to deliver suitable pharmaceutical compositions when the cannular is occluded. At the same time, the number of the passages in the cannular wall is designed to avoid mechanical weakening of the cannular shaft, such as kinking of the cannular shaft.

Particularly, described herein, in certain embodiments, is a transport tube configured to deliver a composition from a source into a subcutaneous space. The transport tube comprises a distal passageway configured to reside in the subcutaneous space. Further, the distal passageway comprises a distal tip that is open when it is first placed into the subcutaneous space. The distal passageway further comprises a plurality of passages connecting a lumen of the transport tube with an outside surface of the transport tube. The plurality of passages is configured to deliver the composition into the subcutaneous space when the distal tip is occluded, thereby allowing the transport tube to reside in the subcutaneous space for at least four days. The transport tube further comprises a proximal solid segment configured to reside on or above a skin surface. Further, the distal passageway and the proximal solid segment are in fluid communication.

The present disclosure provides, in certain embodiments, a transport tube configured to deliver a composition from a source into a subcutaneous space. The transport tube comprises a distal passageway configured to reside in the subcutaneous space. Further, the distal passageway comprises a distal tip that is open when it is first placed into the subcutaneous space. The distal passageway further comprises a plurality of passages connecting a lumen of the transport tube with an outside surface of the transport tube. A passage of the plurality of passages comprises a diameter, that is sufficiently small to prevent a flow of the composition through the passage into the subcutaneous space when the distal tip is open and at the same time is sufficiently large to deliver the composition through the passage into the subcutaneous space during an occlusion of the distal tip. The transport tube further comprises a proximal solid segment configured to reside on or above a skin surface. Further, the distal passageway and the proximal solid segment are in fluid communication.

Further described herein, in certain embodiments, is a transport tube configured to deliver a composition from a source into a subcutaneous space. The transport tube comprises a distal passageway configured to reside in the subcutaneous space. Further, the distal passageway comprises a distal tip that is open when it is first placed into the subcutaneous space. The distal passageway further comprises a plurality of passages connecting a lumen of the transport tube with an outside surface of the transport tube. A wall of the distal passageway comprises an empty fraction sufficiently low to prevent a kink in the transport tube. The transport tube further comprises a proximal solid segment configured to reside on or above a skin surface. Further, the distal passageway and the proximal solid segment are in fluid communication.

Further described herein, in certain embodiments, is a method of creating a through-passage in a transport tube. The method comprises (a) solvating a polymer in a solvent, (b) forming the polymer into a sheet, thereby creating a polymer sheet, (c) evaporating the solvent by raising the temperature of the polymer sheet, (d) making a longitudinal seam in a portion of the polymer sheet in order to create a tubular shape, and (e) creating a through-passage by application of a laser either after (c) or (d).

Also described herein, in certain embodiments, is a method of creating interconnected passages in a transport tube. The method comprises (a) adding a porogen compound to a polymer resin to create a mixture, (b) heating said mixture to above a melting point or glass transition point of the polymer resin, and (c) forcing molten said mixture though a die to create a tubular shape.

Further described herein, in certain embodiments, is a method for delivering a composition to a subcutaneous space in a subject to treat a disease or disorder. The method comprises providing a transport tube, where the transport tube comprises a distal passageway configured to reside in the subcutaneous space and a proximal solid segment configured to reside on or above a skin surface. Further, the distal passageway and the proximal solid segment are in fluid communication. The method further comprises inserting the transport tube into the subcutaneous space and delivering a sufficient amount of the composition through the transport tube into the subcutaneous space. A sufficient amount of the composition is delivered when a distal tip of the distal passageway becomes occluded in the subcutaneous space or when the distal tip is not occluded in the subcutaneous space.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

The terms “coupled” and “connected,” along with their derivatives, may be used herein. It should be understood that these terms are not intended as synonyms for each other. Rather, in particular embodiments, “connected” may be used to indicate that two or more elements are in direct physical or electrical contact with each other. “Coupled” may be used to indicate that two or more elements are in direct physical or electrical contact. However, “coupled” may also be used to indicate that two or more elements are not in direct contact with each other, but yet still cooperate or interact with each other.

As used herein, the term “passage”, “pore”, “macropore”, or “hole” may refer to small hollow spaces in walls of a larger hollow cylinder that allows a composition (e.g., insulin composition, such as those described herein) to escape from the lumen of the larger hollow cylinder (e.g., insulin transport tube). In some examples, the passage connects a lumen of the larger hollow cylinder with an outside surface of the larger hollow cylinder. In some examples, the passage connects with another passage to form interconnected passages. In some examples, the passage does not connect with another passage.

As used herein, the term “tube”, “cannula”, or “passageway” may refer to a hollow cylinder that can be used for transporting or delivery a composition (e.g., insulin composition, such as those described herein). In some examples, the tube comprises a solid wall, a porous wall (e.g., with passages), or a combination thereof.

With respect to the use of any plural and/or singular terms herein, the plural can be translated to the singular and/or the singular can be translated to the plural, as is appropriate to the context and/or application. The various singular/plural permutations may be expressly set forth herein for sake of clarity.

The present disclosure provides methods and devices that allow insulin to flow diffusely through a side wall of an insulin delivery cannula in the event of distal occlusion and, at the same time, minimize the development of a region of wall weakness which would predispose to cannula kinking. In some embodiments, such methods and devices comprise the use of a macroporous cannula material whose passages are sufficiently large to allow the aqueous insulin composition to penetrate, but not so large as to adversely affect mechanical stability.

### Pharmaceutical Composition Delivering Device

A device of the present disclosure comprises a transport tube configured to deliver a composition from an extracorporeal source into a subcutaneous space. In some embodiments, the transport tube comprises a distal passageway configured to reside in the subcutaneous space. The distal passageway comprises a distal tip, which is open when it is first placed into the subcutaneous space. The distal passageway further comprises a plurality of passages connecting a lumen of the transport tube with an outside surface of the transport tube. In some embodiments, the transport tube further comprises a proximal solid segment configured to reside on or above a skin surface. Further, the distal passageway and the proximal solid segment are in fluid communication. In some further cases, the proximal solid segment is configured to reside in the dermis, epidermis, or a combination thereof. In some embodiments, the transport tube is about 6 mm to 18 mm in length. In some cases, the transport tube is about 6 mm to about 15 mm in length. In some cases, the transport tube is about 6 mm to about 12 mm in length. In some cases, the transport tube is about 6 mm to about 9 mm in length. In some instances, the transport tube is about 6 mm to about 9 mm in length. In some instances, the transport tube is about 6 mm to about 7 mm, about 6 mm to about 8 mm, about 6 mm to about 9 mm, about 7 mm to about 8 mm, about 7 mm to about 9 mm, or about 8 mm to about 9 mm in length. In some instances, the transport tube is about 6 mm, about 7 mm, about 8 mm, or about 9 mm in length. In some instances, the transport tube is at least about 6 mm, about 7 mm, or about 8 mm in length. In some instances, the transport tube is at most about 7 mm, about 8 mm, or about 9 mm in length. In some embodiments, the distal passageway is about 4 mm to about 18 mm in length. In some cases, the distal passageway is about 4 mm to about 18 mm in length. In some cases, the distal passageway is about 4 mm to about 8 mm, about 4 mm to about 12 mm, about 4 mm to about 16 mm, about 4 mm to about 18 mm, about 8 mm to about 12 mm, about 8 mm to about 16 mm, about 8 mm to about 18 mm, about 12 mm to about 16 mm, about 12 mm to about 18 mm, or about 16 mm to about 18 mm in length. In some cases, the distal passageway is about 4 mm, about 8 mm, about 12 mm, about 16 mm, or about 18 mm in length. In some cases, the distal passageway is at least about 4 mm, about 8 mm, about 12 mm, or about 16 mm in length. In some cases, the distal passageway is at most about 8 mm, about 12 mm, about 16 mm, or about 18 mm in length. In some embodiments, the proximal solid segment is about 1 mm to 10 mm in length. In some cases, the proximal solid segment is about 1 mm to about 10 mm in length. In some cases, the proximal solid segment is about 1 mm to about 3 mm, about 1 mm to about 6 mm, about 1 mm to about 10 mm, about 3 mm to about 6 mm, about 3 mm to about 10 mm, or about 6 mm to about 10 mm in length. In some cases, the proximal solid segment is about 1 mm, about 3 mm, about 6 mm, or about 10 mm in length. In some cases, the proximal solid segment is at least about 1 mm, about 3 mm, or about 6 mm in length. In some cases, the proximal solid segment is at most about 3 mm, about 6 mm, or about 10 mm in length.

In some embodiments, the transport tube of the present disclosure is configured to connect to an infusion devices. In some cases, the infusion device is configured to be attached to the skin surface of an individual or a subject, with the transport tube of the present disclosure penetrating the skin surface into the subcutaneous space of the individual or the subject. In some cases, the infusion device comprises an external fluid source, such as a syringe, pen (e.g., smart pen), or pump. In some instances, the pump is an on-the-body pump or a patch pump. In some examples, the external fluid source comprises insulin. In some cases, the insulin is a fast-acting insulin. In some cases, the insulin is an intermediate-acting insulin. In some cases, the insulin is a long-acting insulin.

### Passages and Empty Area

The transport tube of the present disclosure comprises a polymer or a resin that can allow passage of a composition. In some embodiments, modifying a polymer in order to allow passage of compositions, such as water, insulin, insulin analog, and/or insulin composition excipients (e.g., phenol, cresol, such as m-cresol, salts, or stabilizing agents) comprises control of several variables. In some embodiments, the variables comprise passage size, empty fraction, passageway interconnectedness, or any combination thereof. In some cases, the passage size comprises the diameter of the individual passages within the material. In some cases, the empty fraction comprises area taken up by passages in cannula walls, stated as a percentage of total wall area. In some cases, the passageway interconnectedness comprises degree to which passages in the wall of the cannula are connected. In some cases, a passage is a conduit between the lumen and the subcutaneous space (e.g., through-passage). In some cases, a passage is connected with another passage within the cannula wall (e.g., interconnected passage).

In some embodiments, the cannula comprises passages that entirely traverse the cannula wall but do not connect with other passages. These passages can be referred to as “through-passages”. In some cases, through-passages are present in a distal passageway of the transport tube configured to reside in the subcutaneous space. In some instances, the through-passages assist in maintaining the flow of a composition (e.g., insulin composition) into the subcutaneous space even when there is some degree of outer surface occlusion by cellular material.

In some embodiments, the cannula comprises is a network of interconnected passages. In some cases, the interconnected passages may be referred to as serpentine passages. In some cases, the interconnected passages are present in a distal passageway of the transport tube configured to reside in the subcutaneous space. In some instances, the interconnected passages assist in maintaining the flow of a composition (e.g., insulin composition) into the subcutaneous space even when there is some degree of outer surface occlusion by cellular material.

In some embodiments, it is important to preserve the columnar stability of the cannula (e.g., in a cannula comprising one or more passages, such as through-passages, interconnected passages, etc.). Columnar stability comprises longitudinal rigidity that limits bending and kinking of the cannula as it is exposed to external bending stresses. Therefore, in such embodiments, the empty fraction (e.g., area taken up by passages in cannula walls, stated as a percentage of total wall area) should not be excessive. In some instances, the empty fraction is also referred to as pore density.

The transport tube may comprise an empty fraction in the distal passageway configured to reside in the subcutaneous space. In some embodiments, the empty fraction is about 2% to about 80%. In some embodiments, the empty fraction is about 2% to about 5%, about 2% to about 10%, about 2% to about 15%, about 2% to about 20%, about 2% to about 25%, about 2% to about 30%, about 2% to about 40%, about 2% to about 50%, about 2% to about 70%, about 2% to about 75%, about 2% to about 80%, about 5% to about 10%, about 5% to about 15%, about 5% to about 20%, about 5% to about 25%, about 5% to about 30%, about 5% to about 40%, about 5% to about 50%, about 5% to about 70%, about 5% to about 75%, about 5% to about 80%, about 10% to about 15%, about 10% to about 20%, about 10% to about 25%, about 10% to about 30%, about 10% to about 40%, about 10% to about 50%, about 10% to about 70%, about 10% to about 75%, about 10% to about 80%, about 15% to about 20%, about 15% to about 25%, about 15% to about 30%, about 15% to about 40%, about 15% to about 50%, about 15% to about 70%, about 15% to about 75%, about 15% to about 80%, about 20% to about 25%, about 20% to about 30%, about 20% to about 40%, about 20% to about 50%, about 20% to about 70%, about 20% to about 75%, about 20% to about 80%, about 25% to about 30%, about 25% to about 40%, about 25% to about 50%, about 25% to about 70%, about 25% to about 75%, about 25% to about 80%, about 30% to about 40%, about 30% to about 50%, about 30% to about 70%, about 30% to about 75%, about 30% to about 80%, about 40% to about 50%, about 40% to about 70%, about 40% to about 75%, about 40% to about 80%, about 50% to about 70%, about 50% to about 75%, about 50% to about 80%, about 70% to about 75%, about 70% to about 80%, or about 75% to about 80%. In some embodiments, the empty fraction is about 2%, about 5%, about 10%, about 15%, about 20%, about 25%, about 30%, about 40%, about 50%, about 70%, about 75%, or about 80%. In some embodiments, the empty fraction is at least about 2%, about 5%, about 10%, about 15%, about 20%, about 25%, about 30%, about 40%, about 50%, about 700%, or about 75%. In some embodiments, the empty fraction is at most about 5%, about 10%, about 15%, about 20%, about 25%, about 30%, about 40%, about 50%, about 70%, about 75%, or about 80%.

The one or more passages (e.g., through-passages or interconnected passages) in the walls of the transport tube (e.g., distal passageway) comprise a mean diameter to allow water droplet permeation in the presence of pressures at or above atmospheric pressure. In some embodiments, the mean diameter is about 5 micrometers to about 200 micrometers. In some embodiments, the mean diameter is about 5 micrometers to about 20 micrometers, about 5 micrometers to about 40 micrometers, about 5 micrometers to about 100 micrometers, about 5 micrometers to about 120 micrometers, about 5 micrometers to about 160 micrometers, about 5 micrometers to about 200 micrometers, about 10 micrometers to about 20 micrometers, about 10 micrometers to about 40 micrometers, about 10 micrometers to about 80 micrometers, about 10 micrometers to about 100 micrometers, about 10 micrometers to about 120 micrometers, about 10 micrometers to about 160 micrometers, about 10 micrometers to about 200 micrometers, about 20 micrometers to about 40 micrometers, about 20 micrometers to about 80 micrometers, about 20 micrometers to about 100 micrometers, about 20 micrometers to about 120 micrometers, about 20 micrometers to about 160 micrometers, about 20 micrometers to about 200 micrometers, about 40 micrometers to about 60 micrometers, about 40 micrometers to about 80 micrometers, about 40 micrometers to about 100 micrometers, about 40 micrometers to about 120 micrometers, about 40 micrometers to about 140 micrometers, about 40 micrometers to about 160 micrometers, about 40 micrometers to about 180 micrometers, about 40 micrometers to about 200 micrometers, about 60 micrometers to about 80 micrometers, about 60 micrometers to about 100 micrometers, about 60 micrometers to about 120 micrometers, about 60 micrometers to about 140 micrometers, about 60 micrometers to about 160 micrometers, about 60 micrometers to about 180 micrometers, about 60 micrometers to about 200 micrometers, about 80 micrometers to about 100 micrometers, about 80 micrometers to about 120 micrometers, about 80 micrometers to about 140 micrometers, about 80 micrometers to about 160 micrometers, about 80 micrometers to about 180 micrometers, about 80 micrometers to about 200 micrometers, about 100 micrometers to about 120 micrometers, about 100 micrometers to about 140 micrometers, about 100 micrometers to about 160 micrometers, about 100 micrometers to about 180 micrometers, about 100 micrometers to about 200 micrometers, about 120 micrometers to about 140 micrometers, about 120 micrometers to about 160 micrometers, about 120 micrometers to about 180 micrometers, about 120 micrometers to about 200 micrometers, about 140 micrometers to about 160 micrometers, about 140 micrometers to about 180 micrometers, about 140 micrometers to about 200 micrometers, about 160 micrometers to about 180 micrometers, about 160 micrometers to about 200 micrometers, or about 180 micrometers to about 200 micrometers. In some embodiments, the mean diameter is about 5 micrometers, about 10 micrometers, about 20 micrometers, about 40 micrometers, about 60 micrometers, about 80 micrometers, about 100 micrometers, about 120 micrometers, about 140 micrometers, about 160 micrometers, about 180 micrometers, or about 200 micrometers. In some embodiments, the mean diameter is at least about 5 micrometers, about 10 micrometers, about 20 micrometers, about 40 micrometers, about 60 micrometers, about 80 micrometers, about 100 micrometers, about 120 micrometers, about 140 micrometers, about 160 micrometers, or about 180 micrometers. In some embodiments, the mean diameter is at most about 10 micrometers, about 20 micrometers, about 40 micrometers, about 60 micrometers, about 80 micrometers, about 100 micrometers, about 120 micrometers, about 140 micrometers, about 160 micrometers, about 180 micrometers, or about 200 micrometers. In some cases, a rising percent empty space assists in delivering insulin quickly by faster liquid transfer through the walls, for example, if the distal opening is occluded. In some instances, this may be the case during large pre-meal boluses. In some cases, the rising empty space compromises tensile strength.

### Compositions and Controlling Movement of Aqueous Compounds

The methods and devices described in the present disclosure can allow for the movement or transport of aqueous compositions. In some embodiments, the composition may comprise an insulin composition. In some cases, the insulin composition comprises water, insulin, an insulin analog, an insulin composition excipient (e.g., phenol, cresol, such as m-cresol, salts, or stabilizing agents), or a combination thereof. In some instances, the insulin composition comprises fast-acting insulin (or rapid-acting insulin) (e.g., lispro, aspart, glulisine, etc.), short-acting insulin (e.g., novolin R, velosulin, etc.), intermediate-acting insulin (e.g., neutral protamine hagedorn (NPH) insulin, etc.), mixed insulin, or long-acting insulin (e.g., glargine insulin, glargine insulin, degludec insulin, detemir insulin, etc.). In some examples, mixed insulin comprises fast-acting insulin (or rapid-acting insulin), short-acting insulin, intermediate-acting insulin, long-acting insulin, or any combination thereof. In some examples, mixed insulin comprises intermediate-acting and short-acting insulin. Examples of mixed insulin comprises, by way of non-limiting example, Humulin 70/30, Novolin 70/30, Novolog 70/30, Humulin 50/50, or Humalog mix 75/25. In some instances, the insulin composition comprises fast-acting insulin, such as those described herein.

In some cases, with regard to biochemical separation of compounds of varying molecular diameters or molecular weights, a particular molecular weight cutoff (MWCO) criterion is specified. The concept with a MWCO membrane comprises that molecules greater in size than the MWCO specification will not pass through the membrane, while those smaller in size will pass through. However, in a system such as an insulin delivery cannula, the flow of water and other highly polar compounds renders the situation more complex, for example, since typical cannula polymer materials are hydrophobic.

In some examples, water vapor exists as individual separated water monomers of exceedingly small diameter. However, in the presence of a hydrophobic medium (e.g., air, lubricious plastic, or oil), liquid water molecules agglomerate with one another due to surface tension and thus form water droplets. In some cases, the size of a liquid water droplet is variable, but is much larger than the molecular size of water. In such cases, the individual water molecules will not leave the droplet to travel through small passages if the passages are located within a hydrophobic medium such as a lubricious polymer, even if the passages are sufficiently large to allow passage of individual water vapor molecules. This concept can be related to the reason that expanded polytetrafluoroethylene (ePTFE) can be effective in “breathing” (e.g., allowing water vapor to pass through but not allowing liquid water to pass through). Accordingly, in some embodiments of the design of a macroporous long-lived insulin delivery cannula as described in the present disclosure, an understanding of molecular-weight cutoff membrane technology is important, but such technology is not be the sole governor of aqueous liquid transfer.

### Tensile Strength

In some embodiments, the transport tube or cannula of the present disclosure is designed so that none of the cannula material is left in the body when it is inserted, when it is being used to deliver a composition, and/or when it is withdrawn. In some cases, the material holds together, maintains sufficient tensile strength, and does not undergo fragmentation, for example, even if occlusive cellular material (e.g., fibrin, platelets, white cell clumps, collagen, etc.) bind to the cannula wall. In some cases, for this reason, weak hydrogels are not good choices for the cannula material. In some cases, the presence of through-passages and interconnected passages can have an adverse effect on tensile strength. In such cases, it is important that the material is a high tensile strength material. Examples of high tensile strength materials include, but are not limited to, polytetrafluoroethylene (PTFE), low- and high-density polyethylene, polyethylene terephthalate, nylon (polyamide), polyimide, polyurethanes with less hydrophilic segments (e.g., less than 10%, 15%, or 20% hydrophilic segments), acrylics, polycarbonates, fluorocarbons (e.g., other than PTFE, such as FEP, PFA, and ETFE), tyrenes, vinyl acetals, vinyl esters, vinylpyridines, or vinylpyrolidones. In some embodiments, the material incorporates hydrogels to some extent. In some cases, hydrophilic compounds (e.g., sodium polyacrylate, pure polyethylene glycol, pure polyethylene oxide, pure polyvinyl alcohol, etc.) are not as suitable as the above-mentioned polymers due to a higher risk for fragmentation, for example, during withdrawal from the subcutaneous tissue.

### Creating Hydrophilicity

If material geometry is equal, it can be more difficult to move water through hydrophobic materials than hydrophilic materials. Therefore, in some cases, it is desirable in hydrophobic materials, to (a) include some hydrophilic domains (e.g., addition of poly(ethylene oxide) (PEO) domains in a block urethane copolymer), and/or (b) treat hydrophobic materials with agents to make the surface more hydrophilic. In some embodiments, the cannula material is exposed to an radio-frequency ionized gas plasma. In some cases, a suitable gas plasma is an oxygen rich plasma. In some instances, oxygen constitutes as least 50% of the feed gas in the oxygen rich plasma. In some embodiments, even without changes in passage size, interconnectedness, and/or distribution, aqueous liquids move through a material faster after a treatment described herein.

Other methods to increase surface hydrophilicity comprise wet chemistry techniques. In some embodiments, the wet chemistry technique comprises depositing a thin layer of polyethylene glycol (PEG) or a derivative thereof on the inner and outer surfaces of a fluoropolymer material. In some cases, a layer of fluoroalkyl-terminated PEG can be applied to the fluoropolymer. See, e.g., Tae, Lammertink et al., “Facile Hydrophilic Surface Modification of Poly(tetrafluoroethylene) Using Fluoroalkyl-Terminated Poly(ethylene glycol)s,” Advanced Materials, Communication Volume 15, Issue 1 2003, incorporated herein by reference in its entirety. In some embodiments, hydrophilicity can be increased using atomic layer deposition. In some cases, atomic layer deposition comprises atomic layer deposition of ZnO. See, e.g., Li, Dongyan et al., “Hydrophilic ePTFE Membranes with Highly Enhanced Water Permeability and Improved Efficiency for Multipollutant Control,” Industrial & Engineering Chemistry Research 2016 55 (10), 2806-2812, incorporated herein by reference in its entirety.

### Polyethersulfone (PES) Materials

PES membrane materials can be used in hemodialysis for separation of plasma compounds and in other separation techniques. See, e.g., Ronco, Claudio et al., “Haemodialysis membranes,” Nat Rev Nephrol 14, 394-410 (2018), incorporated herein by reference in its entirety. In some embodiments, a passage or a plurality of passages can be created within a solid PES material. In some cases, the PES material is quite hydrophobic and liquid aqueous insulin compositions will pass through passages more quickly and efficiently to the extent that the surface can be made hydrophilic. In some instances, hydrophilicity can be increased through sulfuric acid treatment to add molecular sulfonate groups to the PES. In some instances, silicate minerals, such as clay, can be added the PES. In some examples, the addition of sulfonate groups or clay increase water movement through such membranes. See, e.g., Cavalho, T C et al., “Polyethersulfone/clay membranes and its water permeability,” Matéria (Rio J.) [online] 2017, vol. 22, n. 2, e11825. Epub 1 Jun. 2017. ISSN 1517-7076, incorporated herein by reference in its entirety.

The current invention can be distinguished from microporous materials disclosed in Nielsen et al. (U.S. Pat. No. 8,298,172). The disclosure in Nielsen et al. comprises “micropores”, which are small pores designed to allow escape of the very small molecules (e.g., phenol, cresol, or other preservative excipients in insulin compositions) and to prevent the escape of insulin into the subcutaneous space. However, embodiments of the present disclosure comprise insulin escaping into the subcutaneous space to allow exertion of its absorption and its physiological blood glucose-lowering effect. In some embodiments, since the pores in Nielsen et al. prevent escape of insulin (e.g., an insulin hexamer, approximately 50 Angstroms, See, e.g., Weiss et al., “Insulin Biosynthesis, Secretion, Structure, and Structure-Activity Relationships”, in Feingold et al., editors MDText.com, South Dartmouth, MA, February 2014, incorporated herein by reference in its entirety), the cannula wall passageways are, accordingly, exceedingly small. In some further embodiments, the passages should be even smaller in order to avoid escape of monomeric insulin.

### Creating a Cannula

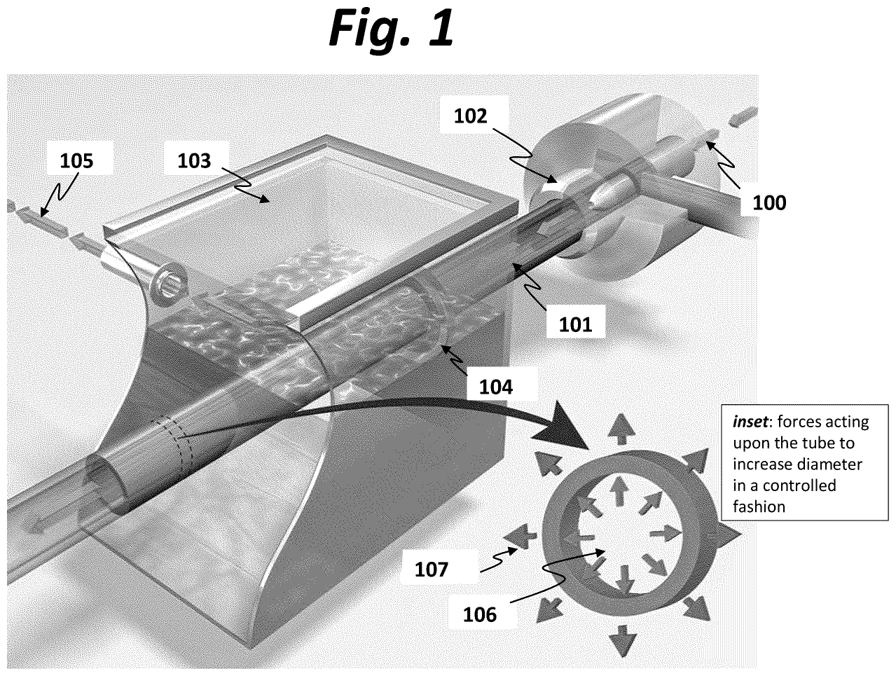

FIG. 1 illustrates a non-limiting example of a method of extruding resin into tubes thereby creating a cannula (e.g., insulin transport tube) in a vacuum tank. In some embodiments, the method comprises controlling tube size (e.g., diameter and wall thickness) in the vacuum tank. As an example, resin is added to a heated hopper to dry the resin. In some cases, the resin has about 1% to about 20% by weight hydrophilic segment. In some cases, the resin has about 1% to about 2%, about 1% to about 5%, about 1% to about 10%, about 1% to about 15%, about 1% to about 20%, about 2% to about 5%, about 2% to about 10%, about 2% to about 15%, about 2% to about 20%, about 5% to about 10%, about 5% to about 15%, about 5% to about 20%, about 10% to about 15%, about 10% to about 20%, or about 15% to about 20% by weight hydrophilic segment. In some cases, the resin has about 1%, about 2%, about 5%, about 10%, about 15%, or about 20% by weight hydrophilic segment. In some cases, the resin has at least about 1%, about 2%, about 5%, about 10%, or about 15% by weight hydrophilic segment. In some cases, the resin has at most about 2%, about 5%, about 10%, about 15%, or about 20% by weight hydrophilic segment. In some examples, the resin has less than 15% by weight hydrophilic segment. In some instances, the hydrophilic segment comprises polyethylene oxide. In some cases, the hydrophilic segment impairs tensile strength. In some instances, the hydrophilic segment impairs tensile strength when over about 5%, about 10%, about 15%, or about 20%. In some examples, the hydrophilic segment impairs tensile strength when over about 15%. In some embodiments, the resin is either loaded manually or by vacuum assist hose into a hopper with an integrated heater (e.g., Thoreson McCosh dryer integrated drying system). In some embodiments, the heating is necessary for drying polymers, especially those such as polyurethane that are hygroscopic (e.g., that tend to take up water).

In some embodiments, a porogen (e.g., NaHCO₃) is placed into the drying hopper. In some cases, the porogen is about 30% to about 80% of the entire mass (porogen plus resin). In some cases, the porogen is about 30% to about 40%, about 30% to about 50%, about 30% to about 60%, about 30% to about 70%, about 30% to about 80%, about 40% to about 50%, about 40% to about 60%, about 40% to about 70%, about 40% to about 80%, about 50% to about 60%, about 50% to about 70%, about 50% to about 80%, about 60% to about 70%, about 60% to about 80%, or about 70% to about 80% of the entire mass (porogen plus resin). In some cases, the porogen is about 30%, about 40%, about 50%, about 60%, about 70%, or about 80% of the entire mass (porogen plus resin). In some cases, the porogen is at least about 30%, about 40%, about 50%, about 60%, or about 70% of the entire mass (porogen plus resin). In some cases, the porogen is at most about 40%, about 50%, about 60%, about 70%, or about 80% of the entire mass (porogen plus resin). In some embodiments, a mixing tool is also integrated into the hopper to fully mix the porogen and the resin.

In some embodiments, FIG. 1 and the method described herein is used to create a tube from a resin or a polymer only. In some alternative embodiments, FIG. 1 and the method described herein is used to create a tube from a resin/porogen mixture.

Then, with the use of a revolving screw, the heated resin (or resin/porogen mixture) is moved toward the extruder crosshead die 102 in a extrusion/auger chamber. In some embodiments, this chamber comprises a stainless steel screw (auger) which propels the resin toward a distal die. In some further embodiments, this chamber comprises one or more heaters (e.g., a series of heaters) that heat the mixture to a temperature to melt the polymer. In some cases comprising a resin/porogen mixture, the heat transforms the porogen to form a gas (e.g., NaHCO₃ turns into water and carbon dioxide gas), so that the gas becomes trapped within the resin and forms passageways.

As the molten resin (or resin/porogen mixture) reaches the heated die, a heated jet of air arrow 100 is forced through the center of the die. As the resin (or resin/porogen mixture) is forced through the die, a tube shape 101 is created. There is positive air pressure inside the tube 101 and low external pressure in the vacuum tank water (see vacuum port 105 in water tank 103). Both of these forces control the dimensions of the tube as shown in the INSET, which shows the internal pressure 106 and the outward force of the vacuum 107. By drawing a vacuum through port 105 to reduce the pressure of the air and water within the water tank 103, a pressure differential that allows air within the extruded tubing to expand outward is created. In some embodiments, this pressure differential is important for size control. In some cases, the tube is passed through a calibration tool 104 (e.g., a ring-shaped device that surrounds the tubing in or near the water bath). In some instances, the calibration tool prevents over-expansion of the tube. In other words, the pressure difference between the vacuum tank and the air inside the tube causes it to expand outward until it is limited by the inner diameter of the calibration tool 104.

In some embodiments, cooling tank allows the transfer of heat away from the hot tube, enabling it to cool properly. In some cases, the hot tube is cooled steadily and gradually. As an example, plastics such as nylon, if cooled quickly, lose burst strength. In some embodiments, the temperature of the water in the cooking tank is adjusted to assist in the regulation of cooling speed. In some embodiments, slow cooling of the tube is achieved by increasing the distance from the extrusion die to the cooling tank so some cooling occurs even before reaching the water cooling tank. In some embodiments, tubing is routed into a conveyor puller from the cooling trough that exerted a controlled pulling force on the tubing. In some cases, the resulting tubing is wound on to a spool.

In some embodiments, further considerations for creating the tubing (or cannula) comprise controlling tubing size parameters. A gauge can be placed outside of the crosshead after the tube is treated with the hot air stream. In some cases, this device (e.g., ultrasonic-based) is placed at a location that has not already undergone substantial cooling. In some instances, the device measures the wall uniformity and wall thickness at multiple points around the circumference. In some examples, the puller can be linked in a servo fashion to this gauge to produce accurate wall thickness control.

In some further embodiments, considerations for creating the tubing (or cannula) comprise the role of a puller. In some cases, extruding a plastic tube is a multi-step process. In some instances, part of the process is regulation of the extrusion speed, which can be partially controlled by the downstream puller (e.g., the last part of the apparatus). In some examples, the puller is part of a computer-controlled servo process and is able to help maintain precise control of wall thickness. In some examples, a cutter is integrated with the puller to cut the tubing to the desired length.

In some further embodiments, considerations for creating the tubing (or cannula) comprise cutting and placing the distal taper on the cannula. Several types of cutters can be used for this purpose. In some cases, for example, a fly-knife cutter is used, which chops through the tubing. In some cases, for example, a planetary cutter is used, which has more of a gentle slicing action. In some embodiments, the distal taper on a cannula is created to allow less trauma and easier insertion into the subcutaneous tissue.

In some alternative embodiments, injection molding is used to make tubes or cannulas. In some cases, the polymer of the tube or cannula is thermoplastic. In some embodiments, reaction injection molding or casting is used for thermosets.

Creation of Passages by Solvent Casting and/or Particle Leaching

Solvent casting comprising use of a porogen can be used to create a passage in polymer sheets and/or polymer membranes. In some embodiments, the solvent may be dissolved once a passage or a plurality of passages are created in the polymer sheets and/or polymer membranes. In some embodiments, solvent casting comprises dispersion of a porogen into a polymer solution. In some cases, the porogen is of a controlled particle size. In some embodiments, the use of a porogen, in accordance with the method described herein, is used to create an interconnected passage or a plurality of interconnected passages in polymer sheets and/or polymer membranes. In some cases, the interconnected passage or the plurality of interconnected passages are created in the distal passageway of the transport tube.

In some embodiments, solute particles are leached or dissolved away by immersing the material in a selective solvent, resulting in the formation of a porous network (e.g., interconnected passages). In some cases, the porogen comprises salt particles, (e.g., NaCl, KCl, KBr, etc.). In some instances, the salt particles, such as NaCl, are inexpensive, widely available, easy to handle, and/or stable under an assortment of processing conditions. In some cases, the porogen may comprise, by way of non-limiting example, sugars, paraffin, poly(N-vinylpyrrolidone), ice particles, gelatin, toluene, hexane, cyclohexanone, 2-ethylhexanol, p-xylene, n-heptane, and/or poly(ethylene glycol). In some further cases, the porogen may comprise inorganic porogens (e.g., silica, sodium hydrogen carbonate (which forms carbon dioxide gas), supercritical carbon dioxide, etc.). In some instances, the porogen (e.g., sugars) are not sufficiently miscible in some bulk solutions. In such instances, more hydrophobic porogens, such as paraffin, hexane, or heptane, etc., can be used.

In some embodiments, passage formation is enhanced when the porogen is non-miscible or only partly miscible in the bulk solution. In some cases, when gases (e.g., supercritical carbon dioxide, supercritical nitrogen, etc.) are used to make passages, the resulting materials can be referred to as foams. In some instances, materials such as polyurethane, polypropylene, and phenolics are made into foamed structures using gases, such as those mentioned above.

In some embodiments, a method of creating interconnected passages in a transport tube comprises (a) adding a porogen compound to a polymer resin to create a mixture, (b) heating said mixture to above a melting point or glass transition point of the polymer resin, and (c) forcing molten said mixture though a die to create a tubular shape. In some cases, the porogen is a gas-forming compound comprising the formula X(HCO₃)_{Y}. In some instances, X is ammonium or an element found in Column I or II of the periodic table of elements. In some instances, Y is 1 if X is ammonium or the element found in Column I, whereas Y is 2 if X is the element found in Column II. In some cases, X is Na and Y is 1. In some cases, the porogen is a non-gas-forming compound or a collection of polymer microspheres.

Creating Passages with Laser Energy and/or Mechanical Means

Application of a controlled energy of a laser beam can be used to create a passage in a polymer sheet and/or polymer membrane. In some embodiments, the laser may comprise, by way of non-limiting example, a yttrium garnet aluminum garnet (YAG) laser, carbon dioxide laser, excimer laser, nonlinear mixing laser, ion laser, neutral atom laser, or any other types of laser. In some embodiments, the laser is used to create a through-passage or a plurality of through-passages in the polymer sheet and/or polymer membrane. In some cases, the through-passage or the plurality of through-passages are created in the distal passageway of the transport tube.

In some further embodiments, mechanical means are employed to create a passage. In some embodiments, mechanical means comprises a small diameter end mill, a wire segment, and/or a drill bit. In some cases, PTFE is stretched biaxially to create expanded PTFE which consists of a unique specialized form of passages, islands, and/or strands. In some embodiments, the mechanical means is used to create a through-passage or a plurality of through-passages in the polymer sheet and/or polymer membrane. In some cases, the through-passage or the plurality of through-passages are created in the distal passageway of the transport tube.

In some embodiments, a method of creating a through-passage in a transport tube comprise (a) solvating a polymer (e.g., polyurethane) in a solvent (e.g., tetrahydrofuran), (b) forming the polymer into a sheet, thereby creating a polymer sheet, (c) evaporating the solvent by raising the temperature of the polymer sheet, (d) making a longitudinal seam in a portion of the polymer sheet in order to create a tubular shape, and (e) creating a through-passage by application of a laser either after (c) or (d). In some cases, the solvent has a boiling point is below 110 degrees C. In some cases, (c) comprises raising the temperature to at least 50 degrees C.

### Rapidly Dissolving Solvents

In some embodiments, mini-explosions are created to make one or more passages in polymer wall. In some cases, mini-explosions are created due to rapid solvent movement out of the polymer walls of the transport tube due to the expansion and evaporation. As an example, polyurethane is solvated in tetrahydrofuran (THF), where the THF makes up between about 5% to about 30% of the total volume, and the solvated polymer is formed into a sheet. In some instances, the more heat that is applied during solvent expansion and evaporation, the faster the vaporized solvent moves through the polymer sheet walls, creating larger and more numerous passages. In some instances, solvents that have a lower vapor pressure (e.g., dimethyl acetamide, dimethyl formamide, dimethyl sulfoxide, etc.) are less suitable for this application due to their lower vapor pressure. In some embodiments, creating predictable size and distribution of passages using these mini-explosions comprises controlling conditions such as the purity, temperature, concentration, and/or dryness of both the polymer and the solvent.

Creating Passages by Adding Porogens Before Extrusion

The addition of porogens (well mixed into the resin) before the materials flows through the extrusion crosshead, followed by exposure to a solvent can allow the porogen material to be dissolved away to form passages. The ideal temperature of the porogen dissolution step can differ depending on the nature of the bulk material, the porogen, and the solvent. In some embodiments, a suitable method to create passages in a polymer comprises choosing a “final” solvent used to extract the porogen that is a poor solvent for the bulk material. As an example, when using a porogen such as NaHCO₃ or a salt such as sodium chloride in a hydrophobic polymer (both of which are soluble in water), a process can be used where the gross structure of the polymer is not affected adversely by exposure to water. In some instances, this may be the case even when heated. In some embodiments, a gas-forming porogen is utilized. In some cases, as the porogen is heated during formation of the tube, carbon dioxide is formed and then departs, leaving interconnected passages in the polymer tube. In some instances, the polymer that contains the porogen must be heated to above the melting point or glass transition temperature of the polymer in order to create passages according to the method described herein. In some embodiments, the gas-forming porogen comprises the chemical structure X(HCO₃)_{Y}, where X refers a cation, such as ammonium, sodium, or any other element found in Column I or II of the periodic table of elements. In some instances, in accordance with the chemical formula, if the charge on X is +1 or if X is an element found in Column I of the periodic table, then Y is 1 (e.g., NH₄(HCO₃), Na(HCO₃), etc.). In some instances, in accordance with the chemical formula, if the charge on X is +2 or if X is an element found in Column II of the periodic table, then Y is 2 (e.g., Ca(HCO₃)₂, Mg(HCO₃)₂, etc.).

In some embodiments, a porogen is a non-gas-forming porogen. In some cases, polymer microspheres are suitable as porogens because they create spherical voids after they are exposed to a solvent that dissolves the microspheres.

Formation of Interconnected Passages in a Polymer

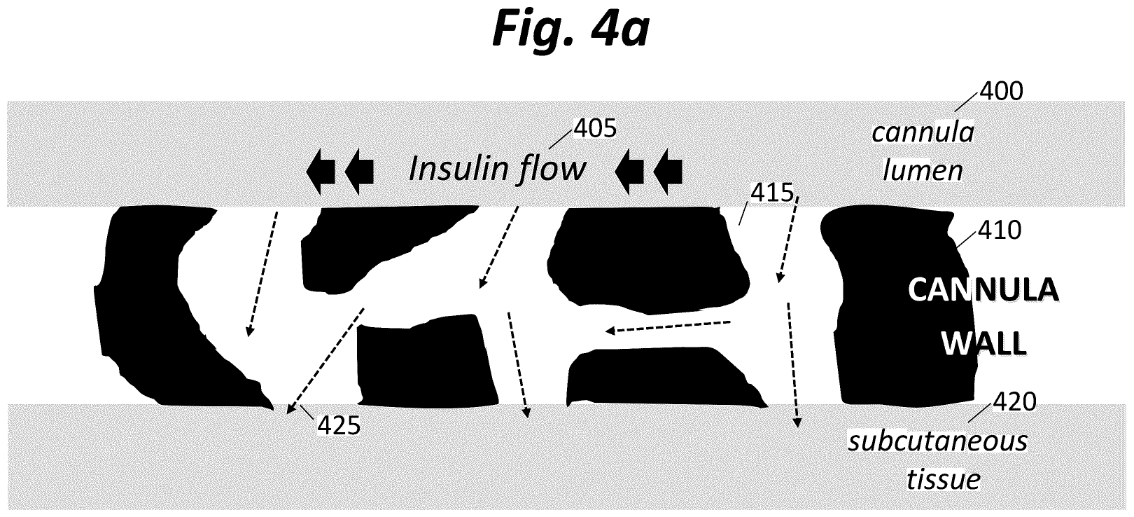

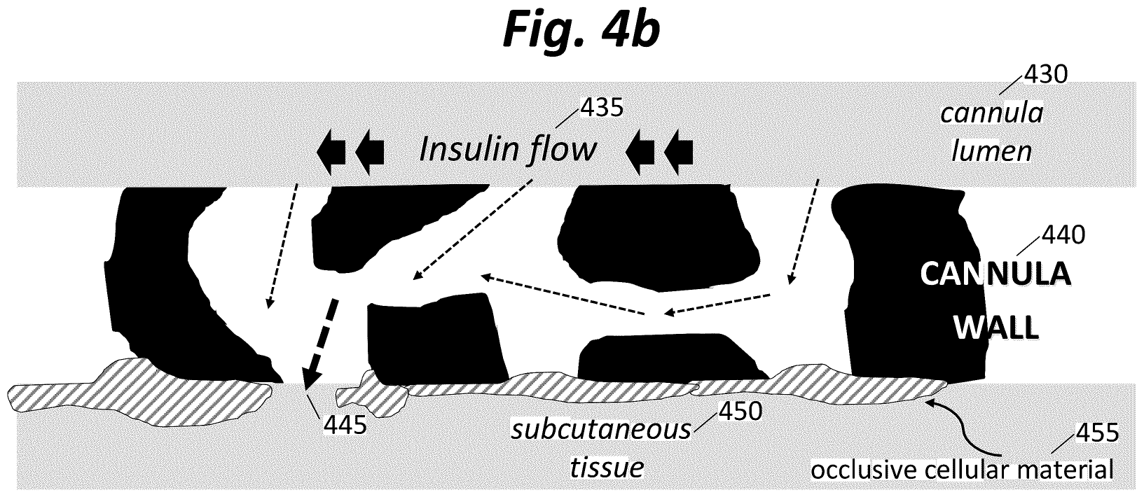

In some embodiments, using NaHCO₃ as a passage-forming compound by the Platkov method leads to different results than if a porogen such as NaCl is used or if laser-ablated through-passages are made. See, e.g., Kaliuzhnyi O., Platkov V., “Formation of Porous Poly(tetrafluoroethylene) Using a Partially Gasified Porogen,” IJMSE. 2020; 17 (2):13-19. In some embodiments, the NaHCO₃ compound when used and heated according to the Platkov method yields a morphology characterized by many thin-walled passages. In some cases, as carbon dioxide is formed, the pressure rises within the wall and some of the boundaries between adjacent passages are broken, yielding a series of interconnected passageways that penetrate through the entire cannula wall. In some cases, use of NaCl leads to a very high porosity and imparts a weakness to the wall structure. In some instances, it leads to substantial degree of water permeability with low interconnectedness and yields a lower tensile strength when compared to the morphology created by NaHCO₃. As illustrated in FIG. 4B, an advantage of the NaHCO₃ method comprises maintenance of insulin flow through the cannula wall due to the presence of interconnected passages within the wall of the cannula (e.g., distal passageway of a transport tube). In some embodiments, interconnectedness is greater when using NaHCO₃ as compared to NaCl or with the use of laser-ablated through-passages. In some embodiments, the use of NaCl is one example of using dissolvable salts. In some cases, other combinations of a cation and an anion can be used. In some instances, the cation is a metal cation. In some examples, the cation is from column I or II of the periodic table (e.g., an alkali metal or alkaline earth metal, such as Li, Na, K, Be, Mg, Ca, etc.) and the anion is from Column VII (or Column 17) (e.g., a halogen, such as F, Cl, Br, I, etc.) of the periodic table which form a dissolvable salt (e.g., KI, CaCl₂), etc.).

In some embodiments, a gas-forming porogen is not limited to NaHCO₃. A more general description of such compounds comprises a chemical structure X(HCO₃)_{Y}. In some cases, X refers to ammonium, sodium, or other element found in Column I or II of the periodic table of elements (e.g., an alkali metal or alkaline earth metal, such as Li, Na, K, Be, Mg, Ca, etc.), as described herein. In some cases, Y is 1 or 2 depending on the charge of X comprising a cation. In some instances, in accordance with the chemical formula, if the charge on X is +1 or if X is an element found in Column I of the periodic table, then Y is 1 (e.g., NH₄(HCO₃), Na(HCO₃), etc.). In some instances, in accordance with the chemical formula, if the charge on X is +2 or if X is an element found in Column II of the periodic table, then Y is 2 (e.g., Ca(HCO₃)₂, Mg(HCO₃)₂, etc.).

In some embodiments, the method described above or variations thereof (e.g., using formation of gas-formed passages) is used to create passages in polymers. Examples of such polymers comprise, by way of non-limiting example, low- and high-density polyethylene, polyethylene terephthalate, nylon (polyamide), polyimide, polyurethanes with less than 15% hydrophilic segments, acrylics, polycarbonates, tyrenes, vinyl acetals, vinyl esters, vinylpyridines, vinylpyrolidones and fluorocarbons such as PTFE, FEP, PFA, ETFE, or any combination thereof.

Creation of Passages and Networks Using Microspheres

In some embodiments, a fabrication technique to create a passage or a plurality of passage (e.g., a network of passages) comprises the use of microspheres. As an example, poly(methyl-methacrylate) (PMMA) microspheres of diameters ranging from about 20 to about 100 micrometers in a water/ethanol mixture (e.g., 30:70 water/ethanol mixture) can be deposited in a solution of PEG dimethacrylate (PEG-DMA). After the network is formed, the PMMA can be removed (dissolved) using acetic acid, leaving a porous network. See, e.g. Christopher J. Bettinger, Robert Langer et al., “Micro- and Nanofabricated Scaffolds,” Principles of Tissue Engineering (Third Edition), 2007, incorporated herein by reference in its entirety.

Creating Passages after Tube Formation

Solid polymer tubes can be formed, as discussed herein, by extruding a tube of controlled size and wall thickness. In some embodiments, a passage is created by mechanical or optical means, as described herein, after the tube is created. In some embodiments, the application of a controlled energy of a laser beam (e.g., yttrium garnet aluminum garnet (YAG) laser, carbon dioxide laser, excimer lasers, nonlinear mixing lasers, ion lasers, neutral atom lasers, etc.) can be used to create a passage. In some embodiments, the wavelength is about 50 nm to about 400 nm. In some embodiments, the wavelength is about 50 nm to about 100 nm, about 50 nm to about 150 nm, about 50 nm to about 200 nm, about 50 nm to about 250 nm, about 50 nm to about 300 nm, about 50 nm to about 350 nm, about 50 nm to about 400 nm, about 100 nm to about 150 nm, about 100 nm to about 200 nm, about 100 nm to about 250 nm, about 100 nm to about 300 nm, about 100 nm to about 350 nm, about 100 nm to about 400 nm, about 150 nm to about 200 nm, about 150 nm to about 250 nm, about 150 nm to about 300 nm, about 150 nm to about 350 nm, about 150 nm to about 400 nm, about 200 nm to about 250 nm, about 200 nm to about 300 nm, about 200 nm to about 350 nm, about 200 nm to about 400 nm, about 250 nm to about 300 nm, about 250 nm to about 350 nm, about 250 nm to about 400 nm, about 300 nm to about 350 nm, about 300 nm to about 400 nm, or about 350 nm to about 400 nm. In some embodiments, the wavelength is about 50 nm, about 100 nm, about 150 nm, about 200 nm, about 250 nm, about 300 nm, about 350 nm, or about 400 nm. In some embodiments, the wavelength is at least about 50 nm, about 100 nm, about 150 nm, about 200 nm, about 250 nm, about 300 nm, or about 350 nm. In some embodiments, the wavelength is at most about 100 nm, about 150 nm, about 200 nm, about 250 nm, about 300 nm, about 350 nm, or about 400 nm. In some alternative embodiments, small diameter end mills, wire segments, or drill bits can be used to create a passage.

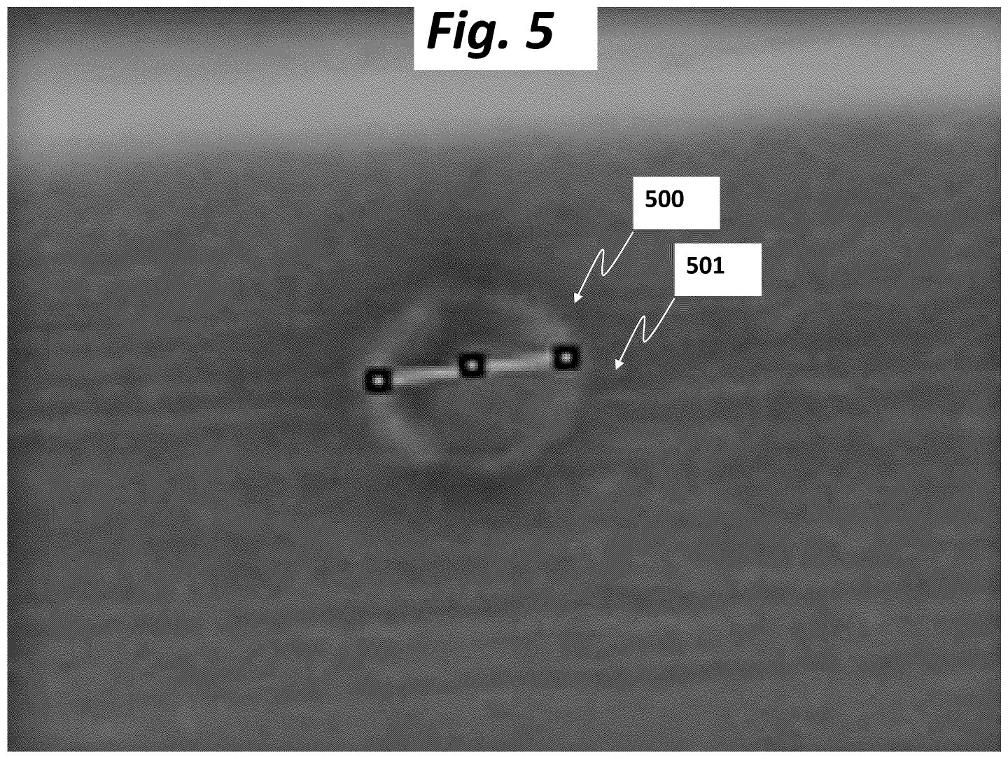

FIG. 5 is a non-limiting example of a photograph of a laser-ablated hole (or passage) 500 in a polyimide tube. In some embodiments, the diameter of the hole is about 40 to 50 micrometers. In some embodiments, the diameter is about 47 micrometers. In some embodiments, the tube is about 0.01 to 0.02 inches in internal diameter. In some embodiments, the tube is about 0.017 inches in internal diameter. In some embodiments, the tube has a wall thickness of about 0.0005 to 0.002 inches. In some embodiments, the tube has a wall thickness of about 0.001 inches. In some embodiments, the laser device used to create the hole comprises a diode-pumped solid state laser. In some embodiments, the laser device comprises an ultraviolet 355 nm diode-pumped solid state laser, Q-switched, N2 assist gas. In some embodiments, to avoid laser ablating the opposite side of the polyimide tube, a small steel cylinder is placed in the lumen of the tube during laser ablation. In some embodiments, the polyimide tube is imaged using a microscope. In some embodiments, the polyimide tube is imaged with a 90× dissecting epiillumination microscope. In some embodiments, the calibration bar 501 is placed in the image by post-processing after the photomicrograph is taken. In some embodiments, the bar indicates the hole diameter.

Forming Sheets into Tubes after Creation of Passages

Sheets of polymer can be manufactured using skiving, molding, solvent evaporation, and/or another process. In some embodiments, a passage is created by mechanical or optical means, as described herein, before the tube is created. In some further embodiments, a passage is created by mechanical or optical means, as described herein, in a polymer sheet. The thickness of the polymer sheet can be any suitable thickness to form a tube or create a passage comprising methods described herein. In some embodiments, the thickness is about 0.01 mm to about 10 mm. In some embodiments, the thickness is about 0.01 mm to about 0.03 mm, about 0.01 mm to about 0.06 mm, about 0.01 mm to about 0.1 mm, about 0.01 mm to about 0.2 mm, about 0.01 mm to about 0.3 mm, about 0.01 mm to about 0.5 mm, about 0.01 mm to about 0.8 mm, about 0.01 mm to about 1 mm, about 0.01 mm to about 3 mm, about 0.01 mm to about 5 mm, about 0.01 mm to about 10 mm, about 0.03 mm to about 0.06 mm, about 0.03 mm to about 0.1 mm, about 0.03 mm to about 0.2 mm, about 0.03 mm to about 0.3 mm, about 0.03 mm to about 0.5 mm, about 0.03 mm to about 0.8 mm, about 0.03 mm to about 1 mm, about 0.03 mm to about 3 mm, about 0.03 mm to about 5 mm, about 0.03 mm to about 10 mm, about 0.06 mm to about 0.1 mm, about 0.06 mm to about 0.2 mm, about 0.06 mm to about 0.3 mm, about 0.06 mm to about 0.5 mm, about 0.06 mm to about 0.8 mm, about 0.06 mm to about 1 mm, about 0.06 mm to about 3 mm, about 0.06 mm to about 5 mm, about 0.06 mm to about 10 mm, about 0.1 mm to about 0.2 mm, about 0.1 mm to about 0.3 mm, about 0.1 mm to about 0.5 mm, about 0.1 mm to about 0.8 mm, about 0.1 mm to about 1 mm, about 0.1 mm to about 3 mm, about 0.1 mm to about 5 mm, about 0.1 mm to about 10 mm, about 0.2 mm to about 0.3 mm, about 0.2 mm to about 0.5 mm, about 0.2 mm to about 0.8 mm, about 0.2 mm to about 1 mm, about 0.2 mm to about 3 mm, about 0.2 mm to about 5 mm, about 0.2 mm to about 10 mm, about 0.3 mm to about 0.5 mm, about 0.3 mm to about 0.8 mm, about 0.3 mm to about 1 mm, about 0.3 mm to about 3 mm, about 0.3 mm to about 5 mm, about 0.3 mm to about 10 mm, about 0.5 mm to about 0.8 mm, about 0.5 mm to about 1 mm, about 0.5 mm to about 3 mm, about 0.5 mm to about 5 mm, about 0.5 mm to about 10 mm, about 0.8 mm to about 1 mm, about 0.8 mm to about 3 mm, about 0.8 mm to about 5 mm, about 0.8 mm to about 10 mm, about 1 mm to about 3 mm, about 1 mm to about 5 mm, about 1 mm to about 10 mm, about 3 mm to about 5 mm, about 3 mm to about 10 mm, or about 5 mm to about 10 mm. In some embodiments, the thickness is about 0.01 mm, about 0.03 mm, about 0.06 mm, about 0.1 mm, about 0.2 mm, about 0.3 mm, about 0.5 mm, about 0.8 mm, about 1 mm, about 3 mm, about 5 mm, or about 10 mm. In some embodiments, the thickness is at least about 0.01 mm, about 0.03 mm, about 0.06 mm, about 0.1 mm, about 0.2 mm, about 0.3 mm, about 0.5 mm, about 0.8 mm, about 1 mm, about 3 mm, or about 5 mm. In some embodiments, the thickness is at most about 0.03 mm, about 0.06 mm, about 0.1 mm, about 0.2 mm, about 0.3 mm, about 0.5 mm, about 0.8 mm, about 1 mm, about 3 mm, about 5 mm, or about 10 mm.

In some embodiments, the application of a controlled energy of a laser beam (e.g., yttrium garnet aluminum garnet (YAG) laser, carbon dioxide laser, excimer lasers, nonlinear mixing lasers, ion lasers, neutral atom lasers, etc.) can be used to create a passage. In some alternative embodiments, small diameter end mills, wire segments, or drill bits can be used to create a passage.

In some embodiments, passages can be created using porogens (e.g., NaHCO₃, NaCl, etc.), followed by dissolution of the porogen material and by creation of a tube. In some embodiments, a tube can be created by wrapping the sheet around a mandrel and welding the polymer, thus creating a seam, so that the sheet forms a desired tube shape. As an example, a laser can be used to impart energy sufficient to weld plastic (make a seam). For meltable thermoplastics (e.g., PE, ABS, nylon, PC, etc.), a nitrogen welder can be used. For thermosets, airless welding can be used, which comprises a process similar to brazing.

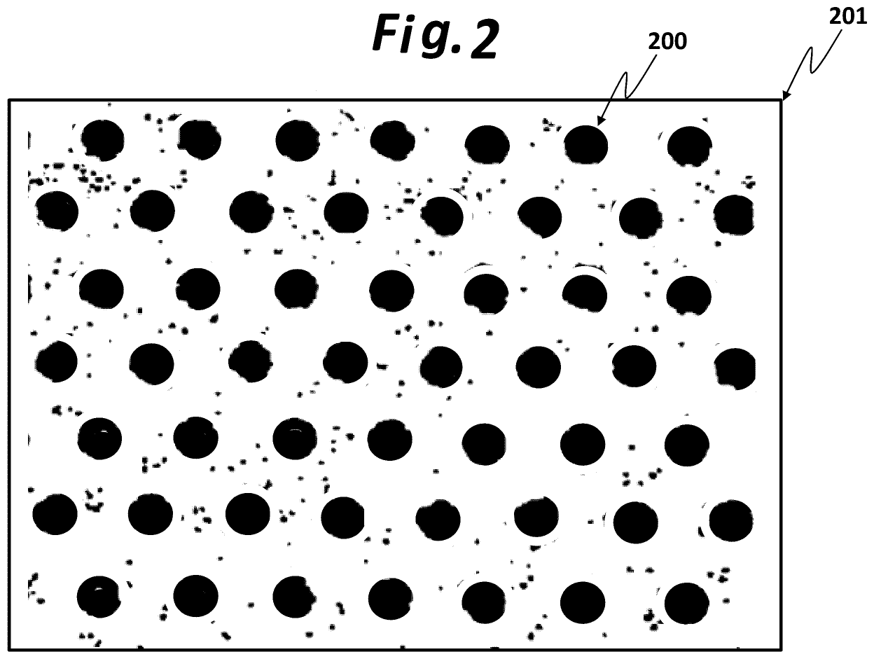

FIG. 2 illustrates a non-limiting example of a polymer sheet with laser-ablated through-passages. This figure comprises a top-view photograph taken through a light microscope. The photograph comprises a sheet of cellulose acetate with a thickness of about 150 micrometers. Straight through-passages were created using an yttrium aluminum garnet (YAG) laser. Passages were formed by a frequency-quadrupled nd:YAG laser (ESI model 4420 Micromachining System, Electro Scientific Industries, Beaverton, Oregon) with a wavelength of 266 nm. The passage diameter for a single passage 200 was approximately 40 μm. Since these passages were larger than m in diameter, the ablation cut out the outline of each passage in a process known as trepanation. As an example, for passages smaller than about 15 μm is diameter, a frequency quadrupled nd:YAG laser can be used to directly ablate the passage rather than ablating the outline. The total area shown by the outline 201 of this sheet segment can be divided by the total passage area to yield the empty fraction. In this case, given a laser-ablated passage diameter of 40 micrometers, each passage has an area of 0.00128 sq mm. FIG. 2 comprises 52 passages in the rectangular sheet. Therefore, the total passage area is 52 passages×0.00128 sq mm, which is about 0.0666 sq mm. The area of the entire rectangular sheet comprises length×height, e.g., 0.80 mm×0.56 mm, which is about 0.448 sq mm. The empty fraction is calculated as the total passage area divided by the total sheet area, e.g., 0.0666/0.448m which is about 15%.

In some embodiment, a mask is used in conjunction with a different kind of laser machine, such as CO₂ laser or excimer laser, to form the passages. In some cases, this technique is used to form a plurality of passages simultaneously, making the process faster.

In some embodiments, in order to maintain the sheets perfectly flat during passage formation, the sheet are adhered to a solid substrate during this process.

In some embodiments, to create a tubular structure, a thermoplastic sheet surrounding a central metal mandrel is welded to make a seam. In some cases, a laser can also be used to make this weld. In some embodiments, the mandrel is then removed. In some cases, to facilitate this removal, a hydrophobic release layer can first be applied to the mandrel.

Passage-Blocking

In some embodiments, the transport tube of the present disclosure comprises a section where the walls do not have a passage. This section may be referred to as a solid tubular segment. In some embodiments, the solid tubular segment comprises the cannula portion that passes through the skin (e.g., epidermis and dermis), and that resides on and/or above the skin. In some cases, if the entire tubular structure (the cannula) has been treated to make passages, the passages are blocked by creating a solid polymer sleeve around the tube. In some instances, the solid polymer sheet around the tube is created by using dip-coating, printing, and/or microdeposition. In the case of a polyurethane cannula with passages, the cannula is grasped by the distal end and dipped it into a solution of polyurethane (e.g., Tecoflex, Tecothane, or similar preparation) dissolved in a solvent (e.g., THF, DMAC, DMF, DMSO, etc.). The solvent is then evaporated slowly. In some case, when using high vapor pressure solvents, such as THF, temperatures over 30 degrees C. are avoided to prevent mini-explosions, as described herein.

In alternative embodiments, cannulas with fluoropolymers comprises an alternative blocking process. In some cases, it is difficult to print, microdeposit, or dip-coat a sleeve on to a fluoropolymer because of its non-stick surface. In such a case, a procedure is employed to first allow the subsequent passage-blocking polymer to adhere to the fluoropolymer. In some instances, a solution known as Tetra-Etch, which is a chemical solution for treating the surface of fluorocarbon to make it bondable to other chemicals, including hydrophilic chemicals, is employed. In such instance, sodium in the Tetra-Etch reacts with highly fluorinated polymers to form a reactive film on the polymer surface. In an alternative case, a fluoropolymer surface is treated with an ionized radio-frequency gas plasma, such as oxygen plasma, to render the surface more hydrophilic and thus bind to a sleeve of a more hydrophilic coating to block the passages in the desired region.

Cannulas in Subcutaneous Tissue

A portion or segment of the transport tube comprising a passage or a plurality of passages is configured to reside in the subcutaneous tissue. In some embodiments, the transport tube comprises a distal passageway comprising the plurality of passages (e.g., through-passages, interconnected passages, etc.) and a solid tubular segment without passages. In some cases, the distal passageway resides in the subcutaneous tissue of an individual or a subject. In some cases, the solid tubular segment resides in the epidermis, dermis, on or above the skin of an individual or a subject, or any combination thereof.

In some embodiments, the plurality of passages (e.g., through-passages, interconnected passages, etc.) in the distal passageway allows a user to keep a cannula in place for longer periods of time. In some embodiments, the cannula can be in place for about 2 days to about 14 days. In some embodiments, the cannula can be in place for about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 12 days, about 2 days to about 14 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 12 days, about 3 days to about 14 days, about 4 days to about 5 days, about 4 days to about 6 days, about 4 days to about 7 days, about 4 days to about 8 days, about 4 days to about 9 days, about 4 days to about 10 days, about 4 days to about 12 days, about 4 days to about 14 days, about 5 days to about 6 days, about 5 days to about 7 days, about 5 days to about 8 days, about 5 days to about 9 days, about 5 days to about 10 days, about 5 days to about 12 days, about 5 days to about 14 days, about 6 days to about 7 days, about 6 days to about 8 days, about 6 days to about 9 days, about 6 days to about 10 days, about 6 days to about 12 days, about 6 days to about 14 days, about 7 days to about 8 days, about 7 days to about 9 days, about 7 days to about 10 days, about 7 days to about 12 days, about 7 days to about 14 days, about 8 days to about 9 days, about 8 days to about 10 days, about 8 days to about 12 days, about 8 days to about 14 days, about 9 days to about 10 days, about 9 days to about 12 days, about 9 days to about 14 days, about 10 days to about 12 days, about 10 days to about 14 days, or about 12 days to about 14 days. In some embodiments, the cannula can be in place for about 2 days, about 3 days, about 4 days, about 5 days, about 6 days, about 7 days, about 8 days, about 9 days, about 10 days, about 12 days, or about 14 days. In some embodiments, the cannula can be in place for at least about 2 days, about 3 days, about 4 days, about 5 days, about 6 days, about 7 days, about 8 days, about 9 days, about 10 days, or about 12 days. In some embodiments, the cannula can be in place for at most about 3 days, about 4 days, about 5 days, about 6 days, about 7 days, about 8 days, about 9 days, about 10 days, about 12 days, or about 14 days.

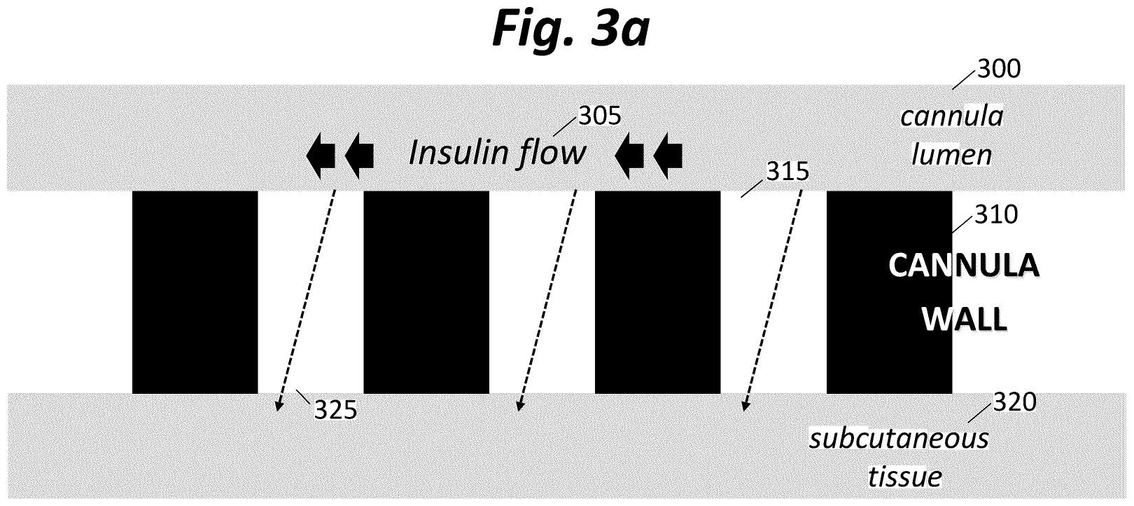

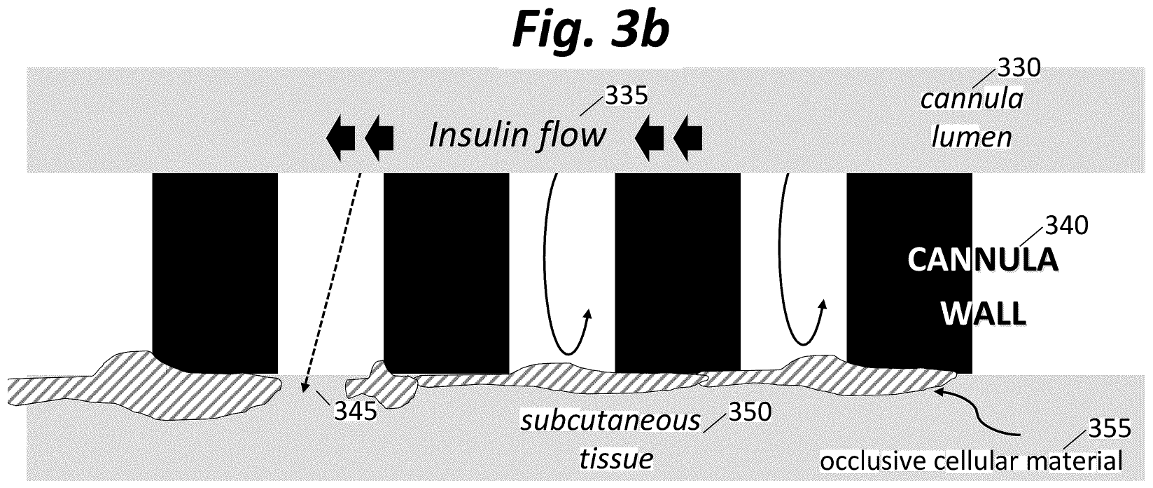

FIG. 3A illustrates a non-limiting example of through-passages in a cannula wall immediately after insertion into subcutaneous tissue. FIG. 3A, FIG. 3B, FIG. 4A, and FIG. 4B are diagrams of tangential cutaway cross-sections of an insulin cannula wall. The insulin composition flows from right to left in the cannula lumen 300 (upper part of each panel) towards a dital tip that is open. The polymer is depicted as black rectangles 310 and the passages (empty areas) are depicted as white rectangles 315 within the polymer. The subcutaneous tissue 320 is at the bottom of the figure. Bulk insulin flow through the lumen of the cannula is shown as heavy short arrows 305.

In FIG. 3A, while the distal tip remains open, the insulin composition flows 315 through the cannula lumen 300. In the presence of an occlusion at the distal tip, insulin will begin to flow through the passages 315 in the cannula wall is depicted as thin dashed lines 325, showing unimpeded flow from the lumen 300 to the subcutaneous space 320. In some embodiments, the cannula is configured to allow little to no insulin composition to flow out of the passages in the absence of an occlusin at the distal tip (e.g., when the distal tip is open).

FIG. 3B illustrates a non-limiting example of impeded flow via through-passages in a cannula wall after at least several days (e.g., about two to seven days) in subcutaneous tissue. After the cannula has been situated in the subcutaneous tissue for a period of time, occlusive cellular material, shown as black hatched lines 355, builds up on the cannula. In some embodiments, the occlusive material blocks the distal tip to prevent insulin flow 335 in the cannula lumen 330. In some embodiments, the occlusive material builds up on the subcutaneous side of the cannula wall 340. In some examples, the material is a blood clot (platelets, blood cells, and/or fibrin), white blood cell clumping, collagen built up due to fibroblast secretion, or other material, or any combination thereof.

In FIG. 3B, the occlusive material blocks some of the through-passages in the cannula, but the insulin composition can continue to flow out of the open through-passage 345 in order to deliver the insulin composition. In some embodiments, due to the morphology of the wall passages (lack of interconnectedness), the insulin flow into the subcutaneous space is reduced compared to the situation immediately after insertion (e.g., as depicted in FIG. 3A).

FIG. 4A illustrates a non-limiting example of interconnected passages in a cannula wall immediately after insertion. The insulin composition flows from right to left in the cannula lumen 400. The polymer is depicted as black shapes 410 and the passages are depicted as white spaces 415 within the polymer. The subcutaneous tissue 420 is at the bottom of the figure. Bulk insulin flow through the lumen of the cannula (upper) is shown as heavy short arrows 405.

In FIG. 4A, while the distal tip remains open, the insulin composition flows 415 through the cannula lumen 400. In the presence of an occlusion at the distal tip, insulin will begin to flow through the passages 415 in the cannula wall is depicted as thin dashed lines 425, showing unimpeded flow from the lumen to the subcutaneous space. In some embodiments, the cannula is configured to prevent the insulin composition to flow out of the passages in the absence of an occlusion at the distal tip (e.g., when the distal tip is open).

FIG. 4B illustrates a non-limiting example of substantial maintenance of flow in interconnected passages in a cannula wall after at least several days (e.g., about two to seven days) in subcutaneous tissue. After the cannula has been situated in the subcutaneous tissue for a period of time, occlusive cellular material, shown as black hatched lines 455, builds up on the cannula. In some embodiments, the occlusive material blocks the distal tip to prevent insulin flow 435 in the cannula lumen 430. In some embodiments, the occlusive material builds up on the subcutaneous side of the cannula wall 440. In some examples, the material is a blood clot (platelets, blood cells, and/or fibrin), white blood cell clumping, collagen built up due to fibroblast secretion, or other material, or any combination thereof.

In FIG. 4B, the occlusive material blocks some of the interconnected passages in the cannula, but the insulin composition can continue to flow out of the open interconnected passage 445 in order to deliver the insulin composition. In some embodiment, because of interconnected passages, even though the occlusive material blocks all but one of the external passage openings, a substantial flow of insulin is maintained as shown by the heavy dashed left lower arrow 445.

### EXAMPLES

The following illustrative examples are representative of embodiments of the software applications, systems, and methods described herein and are not meant to be limiting in any way.

### Example 1—Extrusion of Resin that Contains a Porogen

Thermoplastic polyurethane resin beads are obtained by DSM, Bayer, Lubrizol or other source. The resin had less than 15% by weight hydrophilic segment of polyethylene oxide, which would impair tensile strength when over 15%. The resin was either loaded manually or by vacuum assist hose into a hopper with an integrated heater (e.g., the Thoreson McCosh dryer integrated drying system). The heating was necessary for drying polyurethane, which is hygroscopic (e.g., tend to take up water). Also placed into the drying hopper was the porogen NaHCO₃ in which 30-80% of the entire mass (porogen plus resin) was the porogen. A mixing tool was also integrated into the hopper to fully mix the porogen and the resin.

The flow of the material in the hopper was routed into the extrusion/auger chamber. In this chamber was a stainless steel screw (auger) which propelled the mixed resin/porogen mixture toward a distal die. Also in this chamber were a series of heaters that heated the mixture to the desired temperature necessary to melt the polymer. In this example, when NaHCO₃ was used as the passage-making compound, the heat raised the temperature so that this compound underwent transformation. NaHCO₃ was changed into water and carbon dioxide gas, the latter of which was temporarily trapped within the polymer resin, forming the desired passageways.

An extruder/auger/heater that was suitable for this process was the PAK250 manufactured by Akron-Milacron. The size of the die depended on the desired diameter and wall thickness of the tubing.

After the melted resin plus porogen was forced through the die, the tubing was routed into a cooling trough that had roller bars that held the emerging tubing under the filtered, cooled water. At the distal end of the cooling trough was a laser measuring device with a computer (e.g., a Zumbach monitoring system).

From the cooling trough, the tubing was routed into a conveyor puller that exerted a controlled pulling force on the tubing. The tubing was thus wound on to a spool. In addition to the laser measurement system, the operator checked the diameter and wall thickness with manual metrology devices. Further, samples were also taken to examine porosity, permeability to phosphate-buffered saline, and tensile strength.

### Example 2—Creation of Passages in Polymer Sheet Followed by Welding a Seam

Passages were formed by ablation of a polymer sheet using an energy beam such as a laser or an electron beam. In an experiment, a frequency-quadrupled nd:YAG laser (an ESI model 4420 Micromachining System, Electro Scientific Industries, Beaverton, Oregon) having a wavelength of 266 nm was used for the laser ablating. For passages larger than 15 μm in diameter, ablation was carried out in an orbital pattern for each individual passages (trepanation) to cut out the outline. For passages smaller than about 15 μm in diameter, such as that shown in FIG. 2, a frequency quadrupled nd:YAG laser was used to directly ablate the passage rather than ablating the outline.

### Example 3—Extrusion of Resin without Porogen, and Creating of Passages after Extrusion Using Laser

A passage can be produced in the transport tube (e.g., distal passageway) by sleeve poration. In this method, a passage is created on a tube-shaped substrate. A chemical releasing agent is coated over a mandrel wire, a wire whose diameter is slightly less than the internal diameter. A preformed tube can be placed over the mandrel, followed by laser-machining a passage in the polymer sleeve. This process can be repeated to produce a plurality of passages in the polymer sleeve. The mandrel is then removed.

### Example 4—Creation of Passages in Polymer Tube

FIG. 5 is a photograph of a laser-ablated hole 500 in a polyimide tube. The diameter of the hole was 47 micrometers. The tube was 0.017 inches in internal diameter with 0.001 inch wall thickness. The laser device used to create the hole was an ultraviolet 355 nm diode-pumped solid state laser, Q-switched, N2 assist gas. To avoid laser ablating the opposite side of the polyimide tube, a small steel cylinder was placed in the lumen of the tube during laser ablation. The photo was taken with a 90× dissecting epiillumination microscope. The calibration bar 501 was placed in the image by post-processing after the photomicrograph was taken. The bar indicates the hole diameter, which was measured at 47 micrometers.

### Example 5—Aqueous Flow Through Polyimide Tube into Air

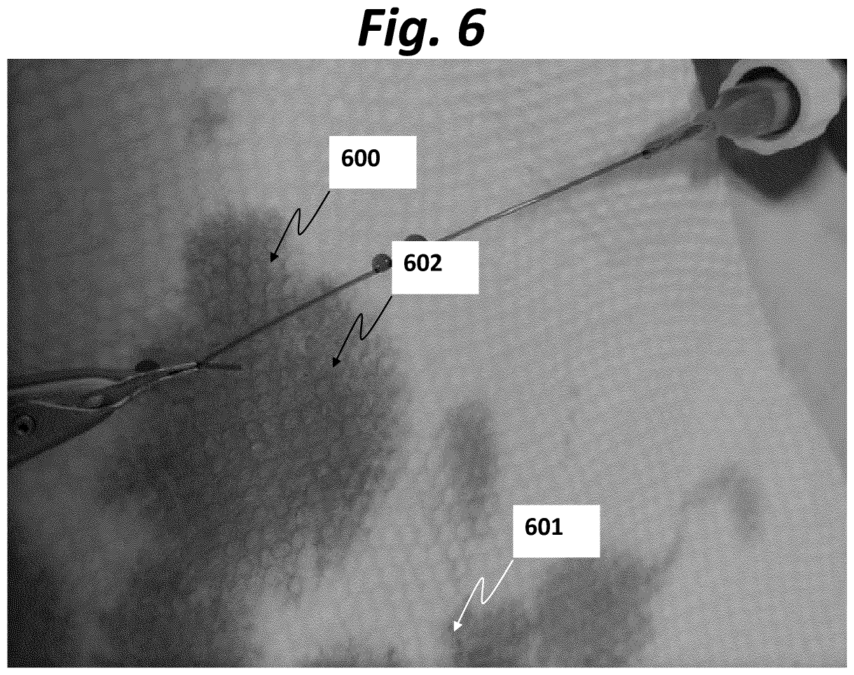

FIG. 6 is a photograph of an experiment in which dyed water is being forced out of a polyimide tube from laser-ablated passages into air. More specifically, the polyimide tube 600 had the terminal end occluded with a clamp 601. The water in the tube was pressurized with air to 1 pound per square inch (PSI). Food coloring dye was added to the water. Two holes were laser-ablated at the sites where dyed water drops 602 were forced into air out of the holes by the elevated water pressure. It should be noted that the internal pressure that an insulin pumps exert within the interior of the cannula lumen without an occlusion is about 1.5-2 PSI during delivery of an insulin microbolus. See, e.g., Dumont-Fillon D, Tahriou H, et al., “Insulin Micropump with Embedded Pressure Sensors for Failure Detection and Delivery of Accurate Monitoring,” Micromachines 2014, 5(4), 1161-1172, incorporated herein in its entirety. Therefore, to be conservative, this experiment was conducted with an internal pressure of 1 PSI. Thus, if the aqueous insulin-like fluid was able to exit from the tube at a pressure of 1 PSI, it certainly would be able to exit at a pressure of 1.5-2 PSI.

### Example 6—Aqueous Flow Through Polyimide Tube into Water

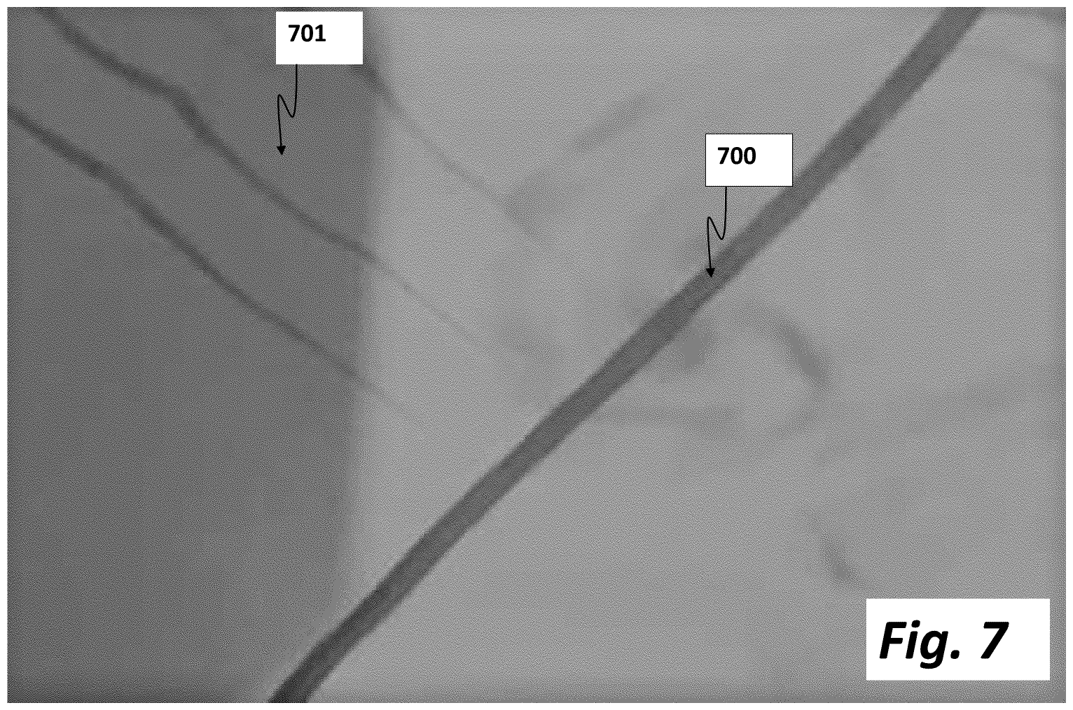

FIG. 7 is a photograph of an experiment in which dyed water is escaping from the interior of a polyimide tube from laser-ablated passages into water during immersion of the tube in water. As in FIG. 6, the end of the tube was clamped during the experiment. This situation (exit of water into a water medium) represents the situation in which insulin is delivered to the subcutaneous interstitial space of an individual (e.g., delivered into an aqueous medium rather than a hydrophobic medium such as air). The photograph shows a tube 700 into which 3 holes of approximately 45 micrometers in diameter were laser-ablated. During an experiment in which the internal pressure of the fluid-filled tube was held at 1 PSI, the dyed water can be seen escaping in three streams 701 into the surrounding water. This experiment demonstrates that a hole of 45 micrometers is sufficient in size to allow water to escape during a pressure that is similar to that obtained in an insulin pump during a microbolus in the situation in which a tube occlusion is not present.

In further experiments, smaller holes that were 20-30 micrometers in diameter were laser-ablated. The results showed no water escaping under the same conditions. When holes of diameter 30-40 micrometers were laser-ablated and the experiment was repeated, small amounts of water were seen to escape, but the flow was not as copious as that seen with holes of at least 40 micrometers.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1.-81. (canceled)

82. A transport tube configured to deliver a composition from a source into a subcutaneous space, the transport tube comprising:
a. a distal passageway configured to reside in the subcutaneous space comprising:
i. a distal tip, wherein the distal tip is open when it is first placed into the subcutaneous space; and
ii. a plurality of passages connecting a lumen of the transport tube with an outside surface of the transport tube, wherein the plurality of passages is configured to deliver the composition from the source into the subcutaneous space when the distal tip is occluded, thereby allowing the transport tube to reside in the subcutaneous space for at least four days; and
b. a proximal solid segment configured to reside on or above a skin surface, wherein the distal passageway and the proximal solid segment are in fluid communication.

83. The transport tube of claim 82, wherein the plurality of passages comprise a plurality of through-passages.

84. The transport tube of claim 83, wherein a through-passage of the plurality of through-passages is not in fluid communication with one another.

85. The transport tube of claim 82, wherein the plurality of passages comprise a plurality of interconnected passages.

86. The transport tube of claim 85, wherein an interconnected passage of the plurality of interconnected passages is in fluid communication with one another.

87. The transport tube of claim 82, wherein the composition comprises insulin.

88. The transport tube of claim 87, wherein the insulin comprises fast-acting insulin, intermediate-acting insulin, or long-acting insulin.

89. The transport tube of claim 82, wherein when the distal tip of the transport tube is occluded in the subcutaneous space, a positive pressure builds up in the lumen of the transport tube.

90. The transport tube of claim 89, wherein the positive pressure in the lumen of the transport tube is at least about 1 pound per square inch (PSI).

91. A transport tube configured to deliver a composition from a source into a subcutaneous space, the transport tube comprising:
a. a distal passageway configured to reside in the subcutaneous space comprising:
i. a distal tip, wherein the distal tip is open when it is first placed into the subcutaneous space; and
ii. a plurality of passages connecting a lumen of the transport tube with an outside surface of the transport tube, wherein a passage of the plurality of passages comprises a diameter, wherein the diameter is sufficiently small to prevent a flow of the composition through the passage into the subcutaneous space when the distal tip is open, and wherein the diameter is sufficiently large to deliver the composition from the source through the passage into the subcutaneous space during an occlusion of the distal tip; and
b. a proximal solid segment configured to reside on or above a skin surface, wherein the distal passageway and the proximal solid segment are in fluid communication.

92. A transport tube configured to deliver a composition from a source into a subcutaneous space, the transport tube comprising:
a. a distal passageway configured to reside in the subcutaneous space comprising:
i. a distal tip, wherein the distal tip is open when it is first placed into the subcutaneous space; and
ii. a plurality of passages connecting a lumen of the transport tube with an outside surface of the transport tube, wherein a wall of the distal passageway comprises an empty fraction sufficiently low to prevent a kink in the transport tube; and
b. a proximal solid segment configured to reside on or above a skin surface wherein the distal passageway and the proximal solid segment are in fluid communication.

93. A method for delivering a composition to a subcutaneous space in a subject to treat a disease or disorder, comprising:
providing a transport tube, wherein the transport tube comprises a distal passageway configured to reside in the subcutaneous space and a proximal solid segment configured to reside on or above a skin surface, wherein the distal passageway and the proximal solid segment are in fluid communication;
inserting the transport tube into the subcutaneous space; and
delivering a sufficient amount of the composition through the transport tube into the subcutaneous space, when a distal tip of the distal passageway becomes occluded in the subcutaneous space or when the distal tip is not occluded in the subcutaneous space.

94. The method of claim 93, wherein the disease or disorder comprises insulin resistance.

95. The method of claim 93, wherein the disease or disorder comprises a Type 1 diabetes mellitus.

96. The method of claim 93, wherein the disease or disorder comprises a Type 2 diabetes mellitus.

97. The method of claim 93, further comprising replacing the transport tube at least 4 days after inserting the transport tube into the subcutaneous space.

98. The method of claim 93, wherein the composition comprises insulin.

99. The method of claim 98, wherein the insulin comprises fast-acting insulin, intermediate-acting insulin, or long-acting insulin.

100. The method of claim 93, wherein the composition further comprises a pharmaceutically acceptable excipient.

101. The method of claim 100, wherein the pharmaceutically acceptable excipient comprises phenol, cresol, a salt, a stabilizing agent, or any combination thereof.
